(19) United States
(12) Patent Application Publication
(10) Pub. No.: US 2023/0183293 A1
(43) Pub. Date: Jun. 15, 2023

(54) METHODS OF TREATING OR PREVENTING CORONAVIRUS INFECTION

(71) Applicant: Aurinia Pharmaceuticals Inc., (US)

(72) Inventors: Y.K. Onno TENG, Leiderdorp (NL); Robert B. HUIZINGA, North Saanich (CA); Neil SOLOMONS, Victoria (CA); Jennifer CROSS, Victoria (CA)

(73) Assignee: Aurinia Pharmaceuticals Inc., Victoria, BC (CA)

(21) Appl. No.: 17/923,552

(22) PCT Filed: May 8, 2021
(86) PCT No.: PCT/IB2021/053922

§ 371 Date: Nov. 4, 2022

(51) Int. Cl. C07K 7/64; A61K 31/5377; A61K 31/573; A61K 9/12; A61P 31/14

(52) U.S. Cl. CPC C07K 7/64; A61K 31/5377; A61K 31/573; A61K 9/12; A61P 31/14

(57) ABSTRACT

Provided herein are methods of using voclosporin for treating or preventing coronavirus infection, particularly in subjects that require immunosuppression.

### CROSS-REFERENCE TO RELATED APPLICATIONS

This application claims priority from U.S. Provisional Application No. 63/021,239, filed May 7, 2020, entitled “METHODS OF TREATING OR PREVENTING VIRUS INFECTION,” and U.S. Provisional Application No. 63/022,357, filed May 8, 2020, entitled “METHODS OF TREATING OR PREVENTING VIRUS INFECTION” the contents of which are incorporated by reference in their entirety.

### FIELD

Provided herein are methods of treating or preventing virus infection, particularly virus infection in subjects that require immunosuppression.

### BACKGROUND

Virus infection can lead to deadly diseases. For example, Coronavirus Disease-2019 (COVID-19), which is caused by severe acute respiratory syndrome coronavirus-2 (SARS-CoV-2), may lead to rapid onset of acute respiratory distress syndrome (ARDS) in addition to causing cardio-pulmonary distress. The spread of COVID-19 is difficult to contain due to the high transmissibility and the lengthy, and often asymptomatic, incubation period.

Certain people are at a substantially increased risk of infection and death during a viral outbreak or pandemic. For example, many patients require intermittent, long-term or even life-long immunosuppression for medical reasons (e.g., due to autoimmune diseases or solid organ transplant). These patients are more susceptible to viral infection due to their immunocompromised state. As such, there is a need for a drug that can maintain patients who require immunosuppression in a healthy state despite their underlying health conditions and provide anti-viral effects at the same time. Provided are embodiments that meet such needs.

### BRIEF SUMMARY

Provided herein are methods of treating or preventing a virus infection in a subject, comprising administering voclosporin to the subject, such as a therapeutically effective amount of voclosporin. In some of any of the provided embodiments, the subject is in need of immunosuppression. In some of any of the provided embodiments, the virus infection is ameliorated by the inhibition of cyclophilin A (CypA) or a CypA associated pathway.

In some of any of the provided embodiments, provided herein is a method of treating or preventing a virus infection in a subject in need of immunosuppression, comprising administering to the subject a therapeutically effective amount of voclosporin, wherein the virus infection is ameliorated by the inhibition of cyclophilin A (CypA) or a CypA associated pathway.

In some of any of the provided embodiments, the virus infection is caused by a virus which is a member of Coronaviridae.

In some of any of the provided embodiments, the virus is an alphacoronavirus, betacoronavirus, deltacoronavirus, or gammacoronavirus.

In some of any of the provided embodiments, the virus is Human coronavirus OC43 (HCoV-OC43), Human coronavirus HKU1 (HCoV-HKU1), Human coronavirus 229E (HCoV-229E), Human coronavirus NL63 (HCoV-NL63), Middle East respiratory syndrome-related coronavirus (MERS-CoV), Severe acute respiratory syndrome coronavirus (SARS-CoV), or Severe acute respiratory syndrome coronavirus 2 (SARS-CoV-2).

In some of any of the provided embodiments, the virus is MERS-CoV, SARS-CoV, or SARS-CoV-2.

In some of any of the provided embodiments, the virus is SARS-CoV-2.

In some of any of the provided embodiments, the therapeutically effective amount is about 0.1 mg/kg/day to about 2 mg/kg/day.

In some of any of the provided embodiments, the therapeutically effective amount is about 7.9 mg BID, about 15.8 mg BID, about 23.7 mg BID, about 31.6 mg BID, about 39.5 mg BID, about 47.4 mg BID, or about 55.3 mg BID.

In some of any of the provided embodiments, the therapeutically effective amount is about 7.9 mg QD, about 15.8 mg QD, about 23.7 mg QD, about 31.6 mg QD, about 39.5 mg QD, about 47.4 mg QD, about 55.3 mg QD, about 63.2 mg QD, about 71.1 mg QD, about 79.0 mg QD, about 86.9 mg QD, about 94.8 mg QD, about 102.7 mg QD, or about 110.6 mg QD.

In some of any of the provided embodiments, the therapeutically effective amount is equivalent to or can achieve a concentration of between about 0.05 μM and about 10 μM, about 0.1 μM and about 5 μM, about 0.2 μM and about 2.5 μM, about 0.3 μM and about 1.0 μM, about 0.4 μM and about 0.9 μM, about 0.5 μM and about 0.8 μM, about 0.1 μM and about 0.5 μM, or about 0.2 μM and about 0.4 μM, or about 0.05, about 0.1, about 0.15, about 0.2, about 0.25, about 0.3, about 0.35, about 0.4, about 0.45, about 0.5, about 0.55, about 0.6, about 0.7, about 0.8, about 0.9, about 1.0, about 1.5, about 2.0, about 2.5, about 3.0, about 3.5, about 4.0, about 4.5, about 5.0, about 6.0, about 7.0, about 8.0, about 9.0, or about 10.0 μM or less.

In some of any of the provided embodiments, the method further comprises monitoring the renal function of the subject.

In some of any of the provided embodiments, monitoring the renal function of the subject comprises:

(a) assessing estimated Glomerular Filtration Rate (eGFR) of the subject at at least a first time point and a second time point on different days; and
(b) (i) if the eGFR of the subject decreases by more than a target % to below a predetermined value between the first and second time points, then reducing the daily dosage or stopping the administering of voclosporin to the subject;

(ii) if the eGFR of the subject decreases by less than the target % between the first and second time points, continuing administering the same daily dosage of voclosporin to the subject.

In some of any of the provided embodiments, the predetermined value is about 50 to about 90 ml/min/1.73 m².

In some of any of the provided embodiments, the predetermined value is about 60 ml/min/1.73 m².

In some of any of the provided embodiments, the target % is about 20% to about 45%.

In some of any of the provided embodiments, the target % is about 20%.

In some of any of the provided embodiments, the subject has an autoimmune disease or a condition associated with transplant rejection.

In some of any of the provided embodiments, the subject has a condition associated with transplant rejection.

In some of any of the provided embodiments, the condition is associated with heart, lung, liver, kidney, pancreas, skin, bowel, or cornea transplant rejection.

In some of any of the provided embodiments, the condition is associated with kidney transplant rejection.

In some of any of the provided embodiments, the subject has an autoimmune disease.

In some of any of the provided embodiments, the therapeutically effective amount of voclosporin is administered without administering a therapeutically effective amount of mycophenolate mofetil (MMF) and/or a therapeutically effective amount of a corticosteroid.

In some of any of the provided embodiments, the method further comprises administering a therapeutically effective amount of mycophenolate mofetil (MMF) and/or a corticosteroid.

In some of any of the provided embodiments, voclosporin is administered by enteral administration (e.g., oral administration, sublingual administration, or rectal administration) or parenteral administration (e.g., intravenous injection, intramuscular injection, subcutaneous injection, intravenous infusion, or inhalation/insufflation).

In some of any of the provided embodiments, voclosporin is administered by enteral administration (e.g., oral administration, sublingual administration, or rectal administration).

In some of any of the provided embodiments, voclosporin is administered by oral administration.

In some of any of the provided embodiments, voclosporin is administered by parenteral administration (e.g., intravenous injection, intramuscular injection, subcutaneous injection, intravenous infusion, or inhalation/insufflation).

In some of any of the provided embodiments, voclosporin is administered by inhalation or insufflation.

In some of any of the provided embodiments, voclosporin is administered in the form of an aerosol.

In some of any of the provided embodiments, voclosporin is administered in a pharmaceutical composition. In some of any of the provided embodiments, the pharmaceutical composition comprises one or more pharmaceutically acceptable excipients. In some of any of the provided embodiments, the pharmaceutically acceptable excipients are independently selected from one or more of comprising alcohol, D-α-tocopherol (vitamin E) polyethylene glycol succinate (TPGS), polysorbate 20 (Tween 20), polysorbate 40 (Tween 40), medium-chain triglycerides, gelatin, sorbitol, glycerin, iron oxide yellow, iron oxide red, titanium dioxide, and water.

### BRIEF DESCRIPTION OF THE DRAWINGS

FIG. 1A shows a cytopathic effect (CPE) reduction assay set-up.

FIG. 1B shows the effect of voclosporin in SARS-CoV-2 CPE reduction assays.

FIG. 1C shows a comparison between the anti-SARS-CoV-2 effect of voclosporin and that of tacrolimus.

FIG. 2A shows the effect of voclosporin in SARS-CoV-2 viral load reduction assay with Vero E6 cells.

FIG. 2B shows the effect of voclosporin in SARS-CoV-2 viral load reduction assay with Calu cells.

FIG. 2C shows the effect of voclosporin on SARS-CoV-2 infected Vero E6 cells. The numbers below the panels indicate the concentration of the testing compound (voclosporin). The fluorescence signal (green fluorescence) indicates viral NSP4 staining (20× objective). Exposure times were the same between the conditions

FIG. 3 shows blood trough levels of voclosporin and tacrolimus in kidney transplant recipients.

FIG. 4 shows a scheme of a study evaluating anti-viral effects of voclosporin in SARS-CoV-2 positive kidney transplant patients.

FIGS. 5A-5E shows the inhibition of SARS-CoV-2 replication by various immunosuppressive drugs and their effects on cell viability of uninfected cells (cytotoxicity) and infected cells (antiviral effect). Voclosporin (FIG. 5A), cyclosporin A (FIG. 5B), everolimus (FIG. 5C), tacrolimus (FIG. 5D), mycophenolate (FIG. 5E).

FIGS. 6A-6D show the impact of cyclosporine A (CsA), tacrolimus (TAC) and voclosporin (VCS) treatment on the production of infectious SARS-CoV-2 progeny by human Calu-3 cells. Experiments were performed using either glass (FIG. 6A and FIG. 6C) or plastic labware (FIG. 6B and FIG. 6D). Cells were infected with SARS-CoV-2 in the presence of different concentrations of VCS, CsA and TAC using stock solutions prepared from pure powders dissolved in DMSO. The viral load in the medium of infected cells was determined by plaque assay on Vero E6 cells using supernatant harvested at 24 h p.i. Viability of uninfected Calu-3 cells treated with the same range of compound concentrations was measured in parallel by a colorimetric viability assay (FIG. 6C; n=12; FIG. 6D; n=3). Mean values±SD are shown and statistical significance of the difference between each concentration and solvent control was assessed by one-way ANOVA. *, p<0.1; **, p<0.01; ***, p<0.001; ****, p<0.0001.

FIGS. 7A-7E show the effects of various compounds on cell viability in a CPE reduction assay with Vero E6 cells infected with SARS-CoV-2. SARS-CoV-2 replication (colored symbols and curves) in Vero E6 cells by various drugs were determined by CPE-reduction assay. For each drug, two-fold serial dilutions of the pharmaceutical formulations were tested. VCS (FIG. 7A), cyclosporine A/Neoral (FIG. 7B), TAC/Prograf (FIG. 7C), EVL/Certican (FIG. 7D), and MMF/Cellcept (FIG. 7E). After preincubation with compound, cells were infected with SARS-CoV-2 and kept in medium containing the drug for 3 days, after which cell viability was measured with a colorimetric assay. Cytotoxicity of the drugs was evaluated in parallel using mock-infected, compound-treated cells (solid grey line). Data points represent the mean±SD of two independent experiments. The CC₅₀ and EC₅₀ were determined by non-linear regression analysis and the regression curves are plotted in the graphs (solid lines).

FIGS. 8A-8B show the inhibition of SARS-CoV-2 replication in Vero E6 cells treated with the a VCS pharmaceutical formulation (FIG. 8A) or placebo (FIG. 8B), as determined by a CPE reduction assay.

FIG. 9 shows the virucidal activity of VCS powder (3.2 μM), a VCS pharmaceutical formulation (3.2 μM), the content of placebo formulations (corresponding to 3.2 μM VCS), and 50% ethanol (positive control) in a plaque assay.

FIGS. 10A-10D show inhibition of SARS-CoV-2 replication by various immunosuppressive compounds in CPE-reduction assays, with stocks prepared from pure compound powders. VCS (FIG. 10A), CsA (FIG. 10B), TAC (FIG. 10C), and MPA (FIG. 10D).

### DETAILED DESCRIPTION

Provided herein are methods of treating or preventing a virus infection in a subject. In some aspects, the methods involve administering voclosporin to the subject, such as a therapeutically effective amount of voclosporin. In some of any of the provided embodiments, the subject is in need of immunosuppression.

Virus infection can lead to deadly results, particularly in vulnerable populations. Between December 2019 and January 2021, severe acute respiratory syndrome coronavirus-2 (SARS-CoV-2), the causative agent of coronavirus disease-2019 (COVID-19), has resulted in over 90 million infections globally. A more severe course of COVID-19 has been correlated to comorbidities commonly present in solid organ transplant recipients (Zhou et al., Lancet. Mar. 28 2020; 395(10229):1054-1062; Huang et al., Lancet. Feb. 15 2020; 395(10223):497-506; Guan et al., Eur Respir J. May 2020; 55(5)doi:10.1183/13993003.00547-2020). Moreover, initial reports showed that the latter are among those at increased risk of COVID-19 related death (Williamson et al., Nature. August 2020; 584(7821):430-436). In particular, these subjects often require intermittent, long-term or even life-long immunosuppression for medical reasons (e.g., due to autoimmune diseases or solid organ transplant), and are at a substantially increased risk of infection and death during a viral infection.

There is a need to find the balance between preventing rejection and controlling infections when prescribing immunosuppressive regimens for transplant recipients. For example, kidney transplant recipients (KTRs) are at increased risk for a more severe course of COVID-19, due to their older age, comorbidity and/or maintenance immunosuppression. Some recommend lowering immunosuppression in KTRs with severe acute respiratory syndrome coronavirus 2 (SARS-CoV-2) infection. The attributable effect of immunosuppression for a more severe course of COVID-19 as well as the most optimal treatment in KTRs is needed. Different reports have shown that immunosuppression did not impose an increased risk for severe COVID-19 disease or mortality (Li et al., J Heart Lung Transplant. May 2020; 39(5):496-497; Zhang et al., Eur Urol. June 2020; 77(6):742-747; Guillen et al., Am J Transplant. July 2020; 20(7):1875-1878; Montagud-Marrahi et al., Am J Transplant. October 2020; 20(10):2958-2959). However, increased death rates have been observed for immunocompromised COVID-19 patients. As the efficacy of vaccines is uncertain in KTRs, methods for treating subjects particularly those in need of immunosuppression, is critically needed. In general, COVID-19 displays a triphasic course: starting with mild flu-like symptoms, followed by a second phase of viral replication and pneumonia, which in a small percentage of cases is followed by a third phase of life-threatening disease, e.g., due to a cytokine storm (Siddiqi et al., J Heart Lung Transplant. May 2020). Antiviral drug treatment is expected to be most effective during earliest stages of disease, while immunosuppressants (e.g. steroids, tocilizumab) may be considered a therapeutic option in later stages of disease to reduce inflammation. An immunosuppressive regimen might ideally prevent rejection, possess antiviral properties and reduce (over)inflammation, whilst still mounting an effective antiviral response to prevent a severe disease course simultaneously. Certain recommendations state lowering but not completely halting immunosuppression and some recommend steroids with CNIs based on advantages observed in vitro.

The current standard for immunosuppressive therapy in most transplant centers consists of a calcineurin inhibitor (CNI), either tacrolimus (TAC) or cyclosporin A (CsA), an antimetabolite agent such as mycophenolate (MPA/MPS) and most often, maintenance steroids. An mTOR inhibitor such as everolimus (EVL) may be prescribed alternatively to MPA or in place of a CNI. The precise impact of immunosuppression on the course of COVID-19 is poorly understood. Early in the disease, (over)immunosuppression might prevent a proper antiviral response, whereas later some immunosuppression might protect against pathological immune overactivation, resulting in less severe disease. Consequently, some recommend to reduce but not completely cede immunosuppression in SARS-CoV-2 infected KTRs, depending on the risk of rejection and disease severity.

Calcineurin inhibitors (CNIs) are cornerstone immunosuppressants in KTRs and some have been reported to possess antiviral activity against RNA viruses. CNIs and mTOR inhibitors such as EVL, in addition to MPA, have been reported to exhibit antiviral activity against human coronaviruses such as SARS-CoV and Middle East respiratory syndrome (MERS-) CoV. Cyclosporin A (CsA) has been shown in vitro to have antiviral effects against a diverse array of RNA viruses including influenza (Ma et al., Antiviral Res. 2016; 133:62-72), hepatitis C (Ishii et al., J Virol. 2006; 80(9):4510-4520), HIV (Braaten et al., J Virol. 1996; 70(8):5170-5176), norovirus (Dang et al, Antimicrobial Agents and Chemotherapy 2017; 61(11):1-17) and SARS-CoV (de Wilde et al., J Gen Virol. 2011; 92:2542-2548; Pfefferle et al., PLoS Pathog 2011; 7(10): 1-15). Without being bound by the theory, the antiviral action of CsA can involve calcineurin-dependent and cyclophilin-dependent mechanisms. In the case of SARS-CoV, the viral Nsp1 protein interacts with cyclophilin A (CypA) to enhance nuclear factor of activated T cells (NFAT)-driven cytokine release in a calcineurin-dependent manner (Pfefferle et al., 2011). However, all in vitro models of CsA and its effects on viral replication show that it only has the potential to be effective at doses that are far in excess of what would be considered safe in humans.

Voclosporin (VCS; also known as LX214 or ISA247) is a novel calcineurin inhibitor (CNI) that is structurally similar to CsA, except for a novel modification of a functional group on the amino acid-1 residue of the molecule, which enhances its binding to calcineurin, and confers better metabolic stability. Voclosporin has been studied in psoriasis, renal organ transplantation, and was recently FDA-approved for treatment of active lupus nephritis in combination with background immunosuppressive therapy. Observations show that VCS is more potent and less toxic at therapeutic levels than other immunosuppressants in its class. Moreover, VCS was shown to inhibit norovirus replication in a CypA-dependent manner and more effectively than CsA. This alteration has changed the binding of voclosporin to calcineurin and has been shown both in vitro and in vivo to increase the binding affinity up to five-fold compared to CsA (Kuglstatter et al., Acta Cryst. 2011; D67:119-23). This modification has also changed the metabolic profile of voclosporin by shifting metabolism away from amino acid-1, which is the major site of metabolism for CsA. The altered metabolic profile has led to a faster elimination of metabolites resulting in lower metabolite exposure compared to CsA. The combination of increased potency and decreased metabolite exposure for voclosporin compared to CsA, has led to better PK/PD relationship, administration of lower doses, and a potentially improved safety profile compared to CsA. Voclosporin has the structure shown below and is disclosed in U.S. Pat. No. 7,332,472, which is incorporated herein by reference in its entirety.

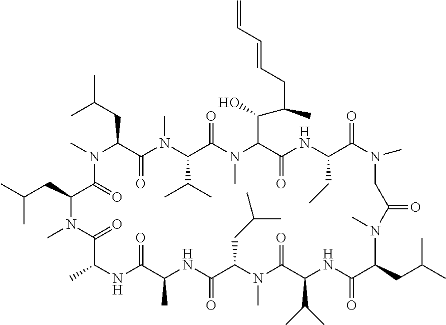 [SMILES: C=C/C=C/C[C@@H](C)[C@@H](O)C1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

Similar to CsA, voclosporin also binds CypA (Kuglstatter et al., Acta Cryst. 2011; D67:119-23). As such, the methods disclosed herein can maintain patients who require immunosuppression in a healthy state despite their underlying health conditions and provide anti-viral effects at the same time.

The provided embodiments are based on the observation as described herein, based on the comparison of the effect of the CNIs, tacrolimus, cyclosporine A, and voclosporin (VCS), as well as other immunosuppressants commonly used in KTRs, on SARS-CoV-2 replication in cell-based assays. As the efficacy of vaccines is uncertain in immunocompromised subjects like KTRs and effective (antiviral) treatment options are limited, finding alternative solutions is crucial to protect these subjects. As demonstrated herein, CNIs demonstrated more potent inhibitory effect of SARS-CoV-2 replication (in cell culture) than other classes of immunosuppressive agents. Strikingly, VCS displayed antiviral activity at 8-fold lower concentrations than TAC. The concentrations of VCS that reduced SARS-CoV-2 viral load can be correlated with tolerable doses in humans that are attainable in KTRs. VCS reduced viral progeny yields in human Calu-3 cells at low micromolar concentrations and did so more effectively than cyclosporin A and tacrolimus. The observations described herein demonstrate the potential benefit of cyclophilin-dependent CNIs, in particular VCS. The results described herein show that VCS exerts a strong inhibitory activity on SARS-CoV-2 replication, even at a low concentration, and demonstrate the utility of VCS in the treatment of viral infections such as COVID-19, in a subject, particularly for subjects in need of immunosuppression. Voclosporin also provide an advantage that it has a higher affinity for calcineurin, and lower nephrotoxicity. Voclosporin can also distribute into organs such as the lungs in higher concentrations than in blood, and higher concentrations are found in red blood cells. Consequently, higher concentrations in specific organs or cells could result in inhibition of the virus. Accordingly, the results support the utility of VCS in treating viral infections, in particular, in KTRs who are at risk of or have been infected with SARS-CoV-2.

In addition, the results described herein show unexpected observations. Pharmaceutical excipients in the preparation of the immunosuppressive compounds showed antiviral effects in the cell-based assays. Unexpectedly, the results were not due to virucidal effects of surfactants that can damage the viral envelope. Highly pure powders of the various immunosuppressive compounds to circumvent the interference caused by excipients in the described antiviral assays demonstrated that excipients that improve solubility and bioavailability of the active compound in pharmaceutical formulations also affect results in cell-based assays. Due to the lipophilic nature of voclosporin and based on the results described herein, the effect of VCS and other compounds were assessed using glass labware (which minimizes binding of VCS to plastic material). The results demonstrated that VCS reduced the production of SARS-CoV-2 infectious progeny in a dose-dependent manner in infected Calu-3 cells, and more effectively than CsA and TAC, and other classes of immunosuppressants, such as EVL and MPA.

### I. Methods of Treatment

Provided herein are methods of treating or preventing a virus infection in a subject, comprising administering voclosporin to the subject. Also provided are uses of voclosporin in treating or preventing a virus infection in a subject. In some aspects, the provided methods or uses include employing a therapeutically effective amount of voclosporin. In some aspects, the subject is in need of immunosuppression. In some aspects, the virus infection is ameliorated by the inhibition of cyclophilin A (CypA) or a CypA associated pathway.

In some embodiments, provided is a method of treating or preventing a virus infection in a subject in need thereof, comprising administering to the subject a therapeutically effective amount of voclosporin, wherein the virus infection is ameliorated by the inhibition of cyclophilin A (CypA) or a CypA associated pathway. In some embodiments, the subject is in need of immunosuppression. In some embodiments, the virus infection is ameliorated by the inhibition of cyclophilin A (CypA). In some embodiments, the virus infection is ameliorated by the inhibition of a CypA associated pathway.

In some aspects, voclosporin (also known as LX214 or ISA247), a therapeutically effective amount thereof, and/or a composition comprising voclosporin, is employed in the provided compositions, methods and uses. Uses include uses of voclosporin or composition comprising the same, in such methods, such as therapeutic methods, and treatments, such as a treatment regimen, and uses of voclosporin or composition comprising the same, in the preparation of a medicament, in order to carry out such therapeutic methods and treatments. Also provided are voclosporin or composition comprising the same for use in treating or preventing a viral infection, reducing viral replication, ameliorating symptoms associated with viral infection, or reducing disease severity or mortality. In some aspect, such uses include performing the methods or treatments as described herein, such as any therapeutic methods or treatment regimens. In some of any of the provided embodiments, voclosporin or a composition comprising the same, is administered as a monotherapy, for example, without administering one or more additional agents. In some of any of the provided embodiments, voclosporin or a composition comprising the same, is administered without administering MMF and/or a corticosteroid. In some of any of the provided embodiments, voclosporin or a composition comprising the same, is administered without administering a therapeutically effective amount of MMF and/or a therapeutically effective amount of a corticosteroid.

In some embodiments, the method or uses are for treating a virus infection or a viral infection. In some embodiments, the method or uses are for preventing a virus infection or a viral infection. In some embodiments, the method comprises treating the virus infection. In some embodiments, the method comprises preventing the virus infection.

In some embodiments, the virus infection is caused by a virus which is a member of Coronaviridae (e.g., alphacoronavirus, betacoronavirus, deltacoronavirus, or gammacoronavirus), Orthomyxoviridae (e.g., an influenza virus), Flaviviridae (e.g., flavivirus or hepacivirus), or Caliciviridae (e.g., norovirus).

In some embodiments, the virus infection is caused by a virus which is a member of Coronaviridae. In some embodiments, the virus is an alphacoronavirus (e.g., HCoV-229E or HCoV-NL63), a betacoronavirus (e.g. HCoV-OC43, HCoV-HKU1, MERS-CoV, SARS-CoV, or SARS-CoV-2), a deltacoronavirus, or a gammacoronavirus.

In some embodiment, the virus is an alphacoronavirus. In some embodiments, the virus is HCoV-229E or HCoV-NL63. In some embodiments, the virus is HCoV-229E. In some embodiments, the virus is HCoV-NL63.

In some embodiments, the virus is a betacoronavirus. In some embodiments, the virus is HCoV-OC43, HCoV-HKU1, MERS-CoV, SARS-CoV, or SARS-CoV-2. In some embodiments, the virus is MERS-CoV, SARS-CoV, or SARS-CoV-2. In some embodiments, the virus is HCoV-OC43. In some embodiments, the virus is HCoV-HKU1. In some embodiments, the virus is HMERS-CoV. In some embodiments, the virus is SARS-CoV. In some embodiments, the virus is SARS-CoV-2.

In some embodiments, the virus is a deltacoronavirus. In some embodiments, the virus is a gammacoronavirus.

In some embodiments, the virus infection is caused by a virus which is a member of Orthomyxoviridae (e.g., an influenza virus).

In some embodiments, the virus infection is caused by a virus which is a member of Flaviviridae. In some embodiments, the virus is a flavivirus. In some embodiments, the virus is a hepacivirus. In some embodiments, the virus is hepacivirus C.

In some embodiments, the virus infection is caused by a virus which is a member of Caliciviridae. In some embodiments, the virus is a norovirus.

### II. Dosage

In some embodiments of the methods and uses provided herein, voclosporin is administered four times a day, three time a day, twice a day, or once a day. In some embodiments, voclosporin is administered four times a day. In some embodiments, voclosporin is administered three times a day. In some embodiments, voclosporin is administered twice a day. In some embodiments, voclosporin is administered once a day.

In some embodiments, daily dosage of voclosporin is about 1 mg to about 250 mg, about 5 mg to about 250 mg, about 10 mg to about 250 mg, about 50 mg to about 250 mg, about 100 mg to about 250 mg, about 150 mg to about 250 mg, about 200 mg to about 250 mg, 1 mg to about 200 mg, about 5 mg to about 200 mg, about 10 mg to about 200 mg, about 50 mg to about 200 mg, about 100 mg to about 200 mg, about 150 mg to about 200 mg, about 1 mg to about 150 mg, about 5 mg to about 150 mg, about 10 mg to about 150 mg, about 50 mg to about 150 mg, about 100 mg to about 150 mg, about 1 mg to about 100 mg, about 5 mg to about 100 mg, about 10 mg to about 100 mg, about 50 mg to about 100 mg, about 1 mg to about 50 mg, about 5 mg to about 50 mg, or about 10 mg to about 50 mg. In some embodiments, daily dosage of voclosporin is about 1 mg, about 5 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg. In some embodiments, daily dosage of voclosporin is at least about 1 mg, at least about 5 mg, at least about 10 mg, at least about 20 mg, at least about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg.

In some embodiments, dosage of voclosporin is about 0.1 mg/kg/day to about 2 mg/kg/day, about 0.5 mg/kg/day to about 2 mg/kg/day, about 1 mg/kg/day to about 2 mg/kg/day, about 1.5 mg/kg/day to about 2 mg/kg/day, about 0.1 mg/kg/day to about 1.5 mg/kg/day, about 0.5 mg/kg/day to about 1.5 mg/kg/day, about 1 mg/kg/day to about 1.5 mg/kg/day, about 0.1 mg/kg/day to about 1.0 mg/kg/day, about 0.5 mg/kg/day to about 1.0 mg/kg/day, or about 0.1 mg/kg/day to about 0.5 mg/kg/day. In some embodiments, dosage of voclosporin is about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 mg/kg/day. In some embodiments, dosage of voclosporin is at least about 0.1, at least about 0.2, at least about 0.3, at least about 0.4, at least about 0.5, at least about 0.6, at least about 0.7, at least about 0.8, at least about 0.9, at least about 1.0, at least about 1.1, at least about 1.2, at least about 1.3, at least about 1.4, at least about 1.5, at least about 1.6, at least about 1.7, at least about 1.8, at least about 1.9, or at least about 2.0 mg/kg/day.

In some embodiments, suitable dosages are in increment of about 7.9 mg. In some embodiments, the dosage of voclosporin is about 7.9 mg QD, about 15.8 mg QD, about 23.7 mg QD, about 31.6 mg QD, about 39.5 mg QD, about 47.4 mg QD, about 55.3 mg QD, about 63.2 mg QD, about 71.1 mg QD, about 79.0 mg QD, about 86.9 mg QD, about 94.8 mg QD, about 102.7 mg QD, or about 110.6 mg QD. In some embodiments, the dosage of voclosporin is about 7.9 mg BID, about 15.8 mg BID, about 23.7 mg BID, about 31.6 mg BID, about 39.5 mg BID, about 47.4 mg BID, or about 55.3 BID.

In some embodiments, the blood trough level is about 25 to about 60 ng/mL. In some embodiments, the blood trough level is about 25, about 30, about 35, about 40, about 45, about 50, about 55, or about 60 ng/mL.

In some embodiments, the therapeutically effective amount is an amount that is equivalent to, can be extrapolated to, can achieve, or can achieve as a Cₘₐₓ of a concentration of between about 0.05 μM and about 10 μM, about 0.1 μM and about 5 μM, about 0.2 μM and about 2.5 μM, about 0.3 μM and about 1.0 μM, about 0.4 μM and about 0.9 μM, about 0.5 μM and about 0.8 μM, about 0.1 μM and about 0.5 μM, or about 0.2 μM and about 0.4 μM. In some embodiments, the therapeutically effective amount is an amount that is equivalent to, can be extrapolated to, can achieve, or can achieve as a Cₘₐₓ of a concentration of about 0.05, about 0.1, about 0.15, about 0.2, about 0.25, about 0.3, about 0.35, about 0.4, about 0.45, about 0.5, about 0.55, about 0.6, about 0.7, about 0.8, about 0.9, about 1.0, about 1.5, about 2.0, about 2.5, about 3.0, about 3.5, about 4.0, about 4.5, about 5.0, about 6.0, about 7.0, about 8.0, about 9.0, or about 10.0 μM or less. In some embodiments, t the therapeutically effective amount is an amount that is equivalent to, can be extrapolated to, can achieve, or can achieve as a Cₘₐₓ of a concentration of about 0.2 μM. In some embodiments, the therapeutically effective amount is an amount that is equivalent to, can be extrapolated to, can achieve, or can achieve as a Cₘₐₓ of a concentration of about 0.3 μM. In some embodiments, t the therapeutically effective amount is an amount that is equivalent to, can be extrapolated to, can achieve, or can achieve as a Cₘₐₓ of a concentration of about 0.4 μM. In some embodiments, t the therapeutically effective amount is an amount that is equivalent to, can be extrapolated to, can achieve, or can achieve as a Cₘₐₓ of a concentration of about 0.5 μM.

A. Dosage Adjustment

In some embodiments of the methods and uses disclosed herein, the method or the treatment further comprises or involves monitoring the renal function of the subject. In some embodiments of a method disclosed herein, the method further comprises monitoring the renal function of the subject. One critical parameter used to assess the desirability of dosage reduction is the estimated Glomerular Filtration Rate (eGFR) using the CKD-EP1 formula or other appropriate method. A decrease in eGFR is a negative side effect that may occur during treatment. If the decrease is too severe, the dosage should be altered.

In some embodiments, monitoring the renal function of the subject comprises:

(a) assessing estimated Glomerular Filtration Rate (eGFR) of the subject at at least a first time point and a second time point on different days; and
(b) (i) if the eGFR of the subject decreases by more than a target % to below a predetermined value between the first and second time points, then reducing the daily dosage or stopping the administering of voclosporin to the subject;

(ii) if the eGFR of the subject decreases by less than the target % between the first and second time points, continuing administering the same daily dosage of voclosporin to the subject.

In some embodiments, the first time point is before the beginning of the treatment, at the beginning of the treatment, or during the treatment. In some embodiments, the first time point is on the first day of the treatment before any administration of voclosporin.

In some embodiments, the predetermined value is about 50 to about 90 ml/min/1.73 m². In some embodiments, the predetermined value is about 50, about 55, about 60, about 65, about 70, about 75, about 80, about 85, or about 90 ml/min/1.73 m².

In some embodiments, the target % is about 20% to about 45%. In some embodiments, the target % is about 20%, about 25%, about 30%, about 35%, about 40%, or about 45%.

B. Routes of Administration

In some embodiments of methods and uses disclosed herein, voclosporin may be administered in any suitable form and by any suitable route that will provide sufficient level of voclosporin for treating or preventing virus infection, such as by enteral administration (e.g., oral administration, sublingual administration, or rectal administration) or parenteral administration (e.g., intravenous injection, intramuscular injection, subcutaneous injection, intravenous infusion, or inhalation/insufflation).

In some embodiments, voclosporin is administered by enteral administration. Exemplary routes of enteral administration include, without limitation, oral administration, sublingual administration, and rectal administration (e.g., through the rectum). In some embodiments, the enteral administration comprises oral administration. In some embodiments, the enteral administration comprises sublingual administration. In some embodiments, the enteral administration comprises rectal administration.

In some embodiments, voclosporin is administered by parenteral administration. Exemplary routes of parenteral administration include, without limitation, intravenous injection, intramuscular injection, subcutaneous injection, intravenous infusion, and inhalation/insufflation. In some embodiments, the parenteral administration comprises intravenous injection. In some embodiments, the parenteral administration comprises intramuscular injection. In some embodiments, the parenteral administration comprises subcutaneous injection. In some embodiments, the parenteral administration comprises intravenous infusion. In some embodiments, the parenteral administration comprises inhalation/insufflation.

In some embodiments, voclosporin is administered by inhalation or insufflation. Exemplary types of preparations for inhalation and/or insufflation include, without limitation, sprays, aerosols, mists, capsules, powders, or cartridges for use in an inhaler or insufflator and solutions/suspensions for nebulization. In some embodiments, voclosporin is administered in the form of an aerosol, a spray, a mist, or a powder. In some embodiments, voclosporin is administered in the form of an aerosol. Examples of various types of devices for administering by inhalation or insufflation include, without limitation, a nebulizer, a metered dose inhaler (MDI), and a dry powder inhaler.

C. Combinations

In some embodiments of the methods and uses provided herein, the method or the treatment also involves administering a therapeutically effective amount of mycophenolate mofetil (MMF) and/or a corticosteroid. In some embodiments of a method disclosed herein, the method also comprises administering a therapeutically effective amount of MMF and/or a corticosteroid. In some embodiments, the method comprises administering a therapeutically effective amount of MMF. In some embodiments, the method comprises administering a therapeutically effective amount of a corticosteroid.

In some embodiments, the method comprises administering voclosporin without a therapeutically effective amount of MMF and/or a therapeutically effective amount of a corticosteroid.

In some embodiments of the methods and uses provided herein, the method or the treatment also involves administering a therapeutically effective amount of an additional anti-viral agent. In some embodiments, the method also comprises administering a therapeutically effective amount of an additional anti-viral agent. In some embodiments, the additional anti-viral agent is remdesivir, lopinavir/ritonavir, IFN-α, lopinavir, ritonavir, penciclovir, galidesivir, disulfiram, darunavir, cobicistat, ASC09F, disulfiram, nafamostat, griffithsin, alisporivir, chloroquine, hydroxychloroquine, nitazoxanide, baloxavir marboxil, oseltamivir, zanamivir, peramivir, amantadine, rimantadine, favipiravir, laninamivir, ribavirin, umifenovir, or any combination thereof. In some embodiments, the anti-viral agent is chloroquine. In some embodiments, the anti-viral agent is hydroxychloroquine. In some embodiments, the anti-viral agent is remdesivir.

### III. Compositions

In some embodiments of the methods and uses provided herein, the method or the treatment involves administering a composition, such as a pharmaceutical composition or a therapeutic composition, comprising voclosporin. In some embodiments, the methods disclosed herein comprises administering a composition comprising voclosporin. In some embodiments, the composition comprises an isomeric mixture of voclosporin and its Z-isoform. In some embodiments, the isomeric mixture comprises at least about 99%, about 98%, about 97%, about 96%, about 95%, about 94%, about 93%, about 92%, about 91%, about 90%, about 80%, about 70%, about 60%, about 50%, about 40%, about 30%, about 20%, or about 10% of voclosporin by weight. In some embodiments, the isomeric mixture comprises at least 95% of voclosporin by weight.

In some embodiments of the provided methods and uses, the composition comprising voclosporin is or comprises a pharmaceutical formulation comprising voclosporin. In some embodiments, the pharmaceutical composition comprising voclosporin comprises one or more pharmaceutically acceptable excipients, buffers, carriers and/or vehicles.

In some embodiments, the composition contains conventional pharmaceutical carriers and excipients appropriate for the type of administration contemplated.

In some embodiments, the compositions that include voclosporin, can be formulated in a pharmaceutically acceptable buffer, such as that containing a pharmaceutically acceptable carrier or vehicle. Generally, the pharmaceutically acceptable carriers or vehicles, such as those present in the pharmaceutically acceptable buffer, can be any known in the art. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds. Pharmaceutically acceptable compositions generally are prepared in view of approvals for a regulatory agency or other agency prepared in accordance with generally recognized pharmacopeia for use in animals and in humans.

Pharmaceutical compositions can include carriers such as a diluent, adjuvant, excipient, or vehicle with which the compound is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin. Water is a typical carrier when the pharmaceutical composition is administered intravenously. Saline solutions and aqueous dextrose and glycerol solutions also can be employed as liquid carriers, particularly for injectable solutions. A composition, if desired, also can contain minor amounts of wetting or emulsifying agents, or pH buffering agents. Typically, the compositions containing the compounds are formulated into pharmaceutical compositions using techniques and procedures well known in the art (see e.g., Ansel Introduction to Pharmaceutical Dosage Forms, Fourth Edition, 1985, 126). Generally, the mode of formulation is a function of the route of administration.

In some embodiments, pharmaceutically acceptable excipients, buffers, carriers and/or vehicles is includes one or more among alcohol, D-α-tocopherol (vitamin E) polyethylene glycol succinate (TPGS), polysorbate 20 (Tween 20), polysorbate 40 (Tween 40), medium-chain triglycerides, gelatin, sorbitol, glycerin, iron oxide yellow, iron oxide red, titanium dioxide, and water. Various formulations of voclosporin mixtures are also described in U.S. Pat. Nos. 7,060,672; 7,429,562 and 7,829,533.

### IV. Patient Population

In some embodiments, the subject to be treated in accordance with the methods and uses provided herein include subjects that is at risk of a viral infection or has contracted a viral infection. In some embodiments, the subject to be treated in accordance with the methods and uses provided herein include subjects in need of immunosuppression. In some aspects, the subject is in need of immunosuppression and is at risk of a viral infection or has contracted a viral infection. In some embodiments, the subject to be treated in accordance with the methods and uses provided herein include subjects in need of immunosuppression, for example, due to a risk of transplant rejection. In some aspects, the subject is a candidate for a transplant, such as an organ transplant, a tissue transplant or a cell transplant, and the subject is in need of immunosuppression.

In some embodiments, the subject involved in the methods disclosed herein has an autoimmune disease or a condition associated with transplant rejection. In some of any of the provided embodiments, the subject is a kidney transplant recipient (KTR).

In some embodiments, the subject has a condition associated with transplant rejection. In some embodiments, the condition is associated with heart, lung, liver, kidney, pancreas, skin, bowel, or cornea transplant rejection. In some embodiments, the condition is associated with heart transplant rejection. In some embodiments, the condition is associated with lung transplant rejection. In some embodiments, the condition is associated with liver rejection. In some embodiments, the condition is associated with kidney transplant rejection. In some embodiments, the condition is associated with pancreas transplant rejection. In some embodiments, the condition is associated with skin transplant rejection. In some embodiments, the condition is associated with bowel transplant rejection. In some embodiments, the condition is associated with cornea transplant rejection.

In some embodiments, the subject has an autoimmune disease. Examples of autoimmune diseases include, without limitation, autoimmune hematological disorders (including e.g. hemolytic anemia, aplastic anemia, pure red cell anemia and idiopathic thrombocytopenia), systemic lupus erythematosus, lupus nephritis, polychondritis, sclerodoma, Wegener granulomatosis, dermatomyositis, chronic active hepatitis, myasthenia gravis, psoriasis, Steven-Johnson syndrome, idiopathic sprue, (autoimmune) inflammatory bowel disease (including e.g. ulcerative colitis and Crohn's disease), endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis, glomerulonephritis (with and without nephrotic syndrome, e.g. including idiopathic nephrotic syndrome or minimal change nephropathy) and juvenile dermatomyositis.

### V. Definitions

As used herein and in the appended claims, the singular forms “a”, “an” and “the” include plural forms, unless the context clearly dictates otherwise.

As used herein, and unless otherwise specified, the terms “about” and “approximately,” when used in connection with doses or amounts, contemplate a dose or amount within 10%, within 5%, within 4%, within 3%, within 2%, within 1%, or within 0.5% of the specified dose or amount

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

The terms “treat,” “treating,” and “treatment” refer to an approach for obtaining beneficial or desired results including clinical results. Beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient.

Also encompassed by “treatment” is a reduction of pathological consequence of the disease or disorder. The methods of this disclosure contemplate any one or more of these aspects of treatment.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

### VI. Exemplary Embodiments

Among the provided embodiments are:

1. A method of treating or preventing a virus infection in a subject, comprising administering to the subject a therapeutically effective amount of voclosporin.

2. The method of embodiment 2, wherein the subject is in need of immunosuppression.

3. The method of embodiment 1 or 2, wherein the virus infection is ameliorated by the inhibition of cyclophilin A (CypA) or a CypA associated pathway.

4. A method of treating or preventing a virus infection in a subject in need of immunosuppression, comprising administering to the subject a therapeutically effective amount of voclosporin, wherein the virus infection is ameliorated by the inhibition of cyclophilin A (CypA) or a CypA associated pathway.

5. A composition comprising voclosporin for use in treating or preventing a virus infection in a subject, wherein the composition comprises a therapeutically effective amount of voclosporin, and is administered to the subject.

6. The composition for use of embodiment 5, wherein the subject is in need of immunosuppression.

7. The composition for use of embodiment 5 or 6, wherein the virus infection is ameliorated by the inhibition of cyclophilin A (CypA) or a CypA associated pathway.

8. A composition comprising voclosporin for use in treating or preventing a virus infection in a subject in need of immunosuppression, wherein the composition comprises a therapeutically effective amount of voclosporin, and is administered to the subject; and wherein the virus infection is ameliorated by the inhibition of cyclophilin A (CypA) or a CypA associated pathway.

9. Use of voclosporin in treating or preventing a virus infection in a subject, wherein the subject is administered a therapeutically effective amount of voclosporin.

10. Use of voclosporin in the manufacture of a medicament for treating or preventing a virus infection in a subject, wherein the medicament comprises a therapeutically effective amount of voclosporin, and is administered to the subject.

11. The use of embodiment 9 or 10, wherein the subject is in need of immunosuppression.

12. The use of any of embodiments 9-11, wherein the virus infection is ameliorated by the inhibition of cyclophilin A (CypA) or a CypA associated pathway.

13. Use of voclosporin in treating or preventing a virus infection in a subject in need of immunosuppression, wherein the subject is administered a therapeutically effective amount of voclosporin; and wherein the virus infection is ameliorated by the inhibition of cyclophilin A (CypA) or a CypA associated pathway.

14. Use of voclosporin in the manufacture of a medicament for treating or preventing a virus infection in a subject in need of immunosuppression, wherein the medicament comprises a therapeutically effective amount of voclosporin, and is administered to the subject; and wherein the virus infection is ameliorated by the inhibition of cyclophilin A (CypA) or a CypA associated pathway.

15. The method, the composition for use, or the use of any one of embodiments 1-14, wherein the virus infection is caused by a virus that is a member of Coronaviridae.

16. The method, the composition for use, or the use of embodiment 15, wherein the virus is an alphacoronavirus, a betacoronavirus, a deltacoronavirus, or a gammacoronavirus.

17. The method, the composition for use, or the use of embodiment 15 or 16 wherein the virus is Human coronavirus OC43 (HCoV-OC43), Human coronavirus HKU1 (HCoV-HKU1), Human coronavirus 229E (HCoV-229E), Human coronavirus NL63 (HCoV-NL63), Middle East respiratory syndrome-related coronavirus (MERS-CoV), Severe acute respiratory syndrome coronavirus (SARS-CoV), or Severe acute respiratory syndrome coronavirus 2 (SARS-CoV-2).

18. The method, the composition for use, or the use of any one of embodiments 15-17, wherein the virus is MERS-CoV, SARS-CoV, or SARS-CoV-2.

19. The method, the composition for use, or the use of any one of embodiments 15-18, wherein the virus is SARS-CoV-2.

20. The method, the composition for use, or the use of any one of embodiments 1-19, wherein the therapeutically effective amount is about 0.1 mg/kg/day to about 2 mg/kg/day.

21. The method, the composition for use, or the use of any one of embodiments 1-20, wherein the therapeutically effective amount is about 7.9 mg BID, about 15.8 mg BID, about 23.7 mg BID, about 31.6 mg BID, about 39.5 mg BID, about 47.4 mg BID, or about 55.3 BID.

22. The method, the composition for use, or the use of any one of embodiment 1-20, wherein the therapeutically effective amount is about 7.9 mg QD, about 15.8 mg QD, about 23.7 mg QD, about 31.6 mg QD, about 39.5 mg QD, about 47.4 mg QD, about 55.3 mg QD, about 63.2 mg QD, about 71.1 mg QD, about 79.0 mg QD, about 86.9 mg QD, about 94.8 mg QD, about 102.7 mg QD, or about 110.6 mg QD.

23. The method, the composition for use, or the use of any one of embodiment 1-20, wherein the therapeutically effective amount is equivalent to or can achieve a concentration of between about 0.05 μM and about 10 μM, about 0.1 μM and about 5 μM, about 0.2 μM and about 2.5 μM, about 0.3 μM and about 1.0 μM, about 0.4 μM and about 0.9 μM, about 0.5 μM and about 0.8 μM, about 0.1 μM and about 0.5 μM, or about 0.2 μM and about 0.4 μM, or about 0.05, about 0.1, about 0.15, about 0.2, about 0.25, about 0.3, about 0.35, about 0.4, about 0.45, about 0.5, about 0.55, about 0.6, about 0.7, about 0.8, about 0.9, about 1.0, about 1.5, about 2.0, about 2.5, about 3.0, about 3.5, about 4.0, about 4.5, about 5.0, about 6.0, about 7.0, about 8.0, about 9.0, or about 10.0 μM or less.

24. The method, the composition for use, or the use of any one of embodiments 1-23, wherein the renal function of the subject is monitored.

25. The method, the composition for use, or the use of embodiment 24, wherein monitoring the renal function of the subject comprises:

(a) assessing estimated Glomerular Filtration Rate (eGFR) of the subject at at least a first time point and a second time point on different days; and

(b) (i) if the eGFR of the subject decreases by more than a target % to below a predetermined value between the first and second time points, then reducing the daily dosage or stopping the administering of voclosporin to the subject;

(ii) if the eGFR of the subject decreases by less than the target % between the first and second time points, continuing administering the same daily dosage of voclosporin to the subject.

26. The method, the composition for use, or the use of embodiment 25, wherein the predetermined value is about 50 to about 90 ml/min/1.73 m2.

27. The method, the composition for use, or the use of embodiment 25 or 26, wherein the predetermined value is about 60 ml/min/1.73 m2.

28. The method, the composition for use, or the use of any one of embodiments 25-27, wherein the target % is about 20% to about 45%.

29. The method, the composition for use, or the use of any one of embodiments 25-28, wherein the target % is about 20%.

30. The method, the composition for use, or the use of any one of embodiments 1-29, wherein the subject has an autoimmune disease or a condition associated with transplant rejection.

31. The method, the composition for use, or the use of any one of embodiments 1-30, wherein the subject has a condition associated with transplant rejection.

32. The method, the composition for use, or the use of embodiment 30 or 31, wherein the condition is associated with heart, lung, liver, kidney, pancreas, skin, bowel, or cornea transplant rejection.

33. The method, the composition for use, or the use of any one of embodiments 30-32, wherein the condition is associated with kidney transplant rejection.

34. The method, the composition for use, or the use of embodiment 30, wherein the subject has an autoimmune disease.

35. The method, the composition for use, or the use of any one of embodiments 1-34, wherein the therapeutically effective amount of voclosporin is administered without administering a therapeutically effective amount of mycophenolate mofetil (MMF) and/or a corticosteroid.

36. The method, the composition for use, or the use of any one of embodiments 1-34, further comprising administering a therapeutically effective amount of mycophenolate mofetil (MMF) and/or a therapeutically effective amount of a corticosteroid.

37. The method, the composition for use, or the use of any one of embodiments 1-36, wherein voclosporin is administered by enteral administration, oral administration, sublingual administration, or rectal administration, parenteral administration, intravenous injection, intramuscular injection, subcutaneous injection, intravenous infusion, or inhalation/insufflation.

38. The method, the composition for use, or the use of embodiment 37, wherein voclosporin is administered by enteral administration, oral administration, sublingual administration, or rectal administration.

39. The method, the composition for use, or the use of embodiment 37 or 38, wherein voclosporin is administered by oral administration.

40. The method, the composition for use, or the use of embodiment 37, wherein voclosporin is administered by parenteral administration, intravenous injection, intramuscular injection, subcutaneous injection, intravenous infusion, or inhalation/insufflation.

41. The method, the composition for use, or the use of embodiment 37 or 40 wherein voclosporin is administered by inhalation or insufflation.

42. The method, the composition for use, or the use of embodiment 41, wherein voclosporin is administered in the form of an aerosol.

43. The method, the composition for use, or the use of any one of embodiments 1-42, wherein voclosporin is administered in a pharmaceutical composition.

44. The method, the composition for use, or the use of embodiment 43, wherein the pharmaceutical composition comprises one or more pharmaceutically acceptable excipients.

45. The method, the composition for use, or the use of embodiment 44, wherein the pharmaceutically acceptable excipients are independently selected from one or more of comprising alcohol, D-α-tocopherol (vitamin E) polyethylene glycol succinate (TPGS), polysorbate 20 (Tween 20), polysorbate 40 (Tween 40), medium-chain triglycerides, gelatin, sorbitol, glycerin, iron oxide yellow, iron oxide red, titanium dioxide, and water.

46. The method, the composition for use, or the use of any one of embodiments 1-45, wherein the viral load is reduced in the subject following administration of voclosporin.

47. The method, the composition for use, or the use of any one of embodiments 1-46, wherein the survival of the subject is extended following administration of voclosporin.

### VII. Examples

The following examples are included for illustrative purposes only and are not intended to limit the scope of the invention.

### Example 1

In order to investigate candidate compounds for inhibiting cytopathic effect (CPE) of SARS-CoV-2, Vero E6 cells (African green monkey kidney epithelial cells) were preincubated with voclosporin, cyclosporine A (CsA), or tacrolimus before infection with SARS-CoV-2, and subsequently assessed for viability.

### Method

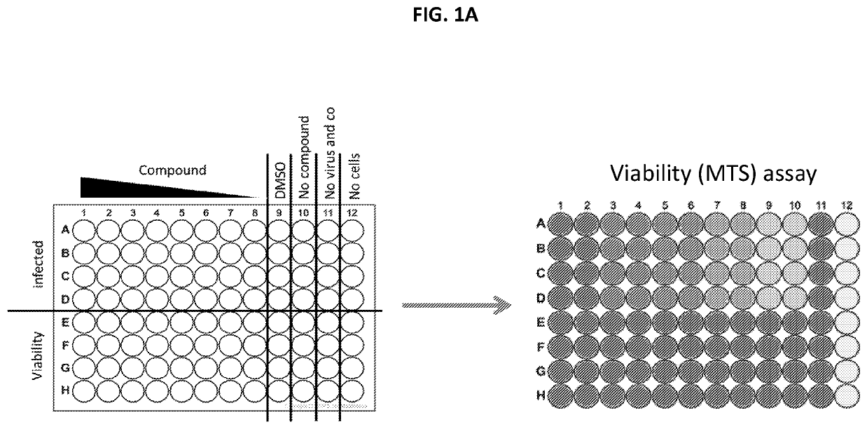

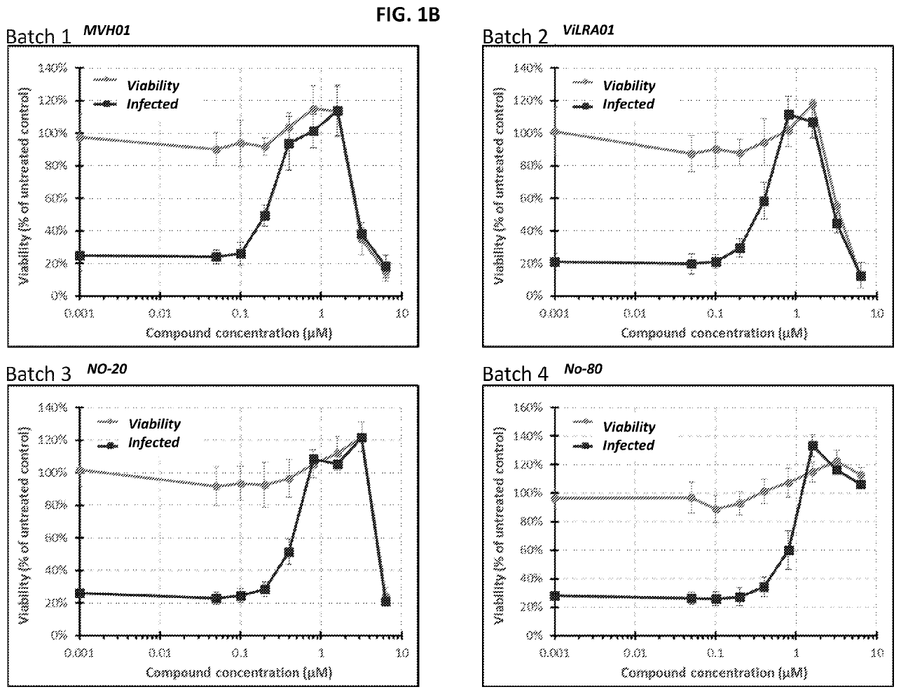

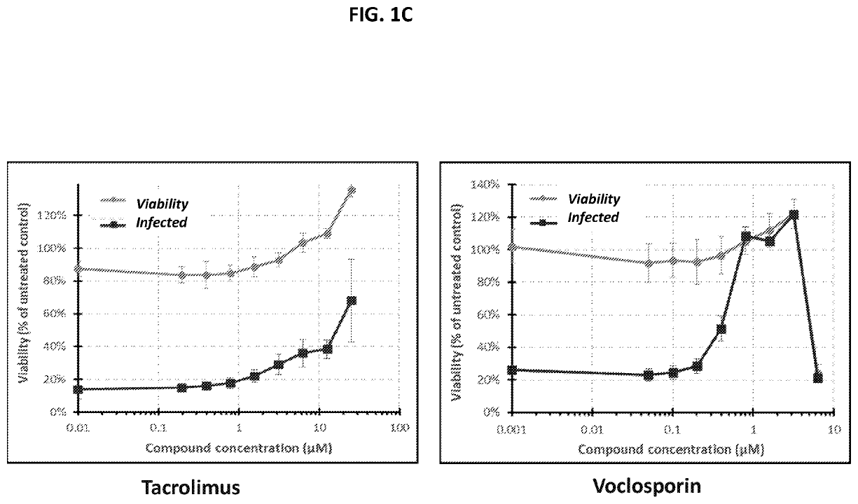

Vero E6 cells were grown in 96 well-plates, and preincubated 60 minutes with media, vehicle and either voclosporin (0.8-100 μM), CsA (0.8-100 μM) or tacrolimus (0.8-100 μM). The cells were then left uninfected or infected with SARS-CoV-2 at multiplicity of infection (MOI) of 0.015, in the presence of the respective compounds at the indicated concentration for 60 minutes. Subsequently, virus was removed from the media, the cells were washed with PBS and were further incubated with fresh medium with the respective compounds, until untreated infected control cells displayed full CPE (3 days). At the end of incubation, the cells were subjected to an MTS viability assay, and subsequently fixed before absorbance analysis in a plate reader (FIG. 1A). The viability of the cells was assessed using an MTS assay to determine both compound cytotoxicity (uninfected cells) and viral cytotoxicity.

### Results

Tacrolimus did not protect the infected cells from virus-induced cytopathic effects, this was apparent using dosages up to 25 μM, after which compound-related cytotoxicity was observed. Alternatively protective effects of CsA occurred between 1.6-12 μM, after which the compound was cytotoxic. Treatment of infected cells with 0.8 μM voclosporin provided viral protection at the same level of cell viability as the mock-infected control.

As shown in FIG. 1B, voclosporin did not display toxicity to uninfected cells at concentrations under 2 and batch 4 voclosporin did not display toxicity to uninfected cells at concentrations up to 10 μM. On the other hand, all tested batches of voclosporin facilitated inhibition of CPE of SARS-CoV-2 at concentrations of 0.01 μM to 1 μM in a dose-dependent manner, as shown by the increase in viability of infected cells. For all batches of voclosporin, treatment at 0.8 μM was efficacious in preserving viability of cells to levels seen for uninfected cells.

As shown in FIG. 1C, voclosporin was able to inhibit SARS-CoV-2 at much lower concentrations (EC₅₀ of ˜0.4 μM) compared to tacrolimus (EC₅₀ of ˜25 μM). In addition, tacrolimus treatment led to compound-related cytotoxicity at concentrations above 25 μM (data not shown). These results indicated that voclosporin displayed in vitro inhibition against SARS-CoV-2 at concentration ranges that did not affect viability of the tested cells.

### Example 2

In order to study the effect of voclosporin on SARS-CoV-2 in vitro, Vero E6 cells or Calu cells (human bronchial airway epithelial cells) were preincubated with voclosporin before infection with SARS-CoV-2, and subsequently harvested to determine viral load by plaque assays.

### Methods

Vero E6 cells and Calu were grown in 96 well-plates, and were preincubated with 0.01 μM to 10.00 μM of voclosporin for 60 minutes. The cells were then infected with SARS-CoV-2 at MOI of 1, in the presence of voclosporin at the indicated concentration for 60 minutes. Subsequently, virus was removed from the media, the cells were washed with PBS and were further incubated with fresh medium with voclosporin at the indicated concentration for 16 hours. At the end of incubation, the culture medium was harvested to determine viral load by plaque assay. To illustrate the amount of infection, the respective cells were also subjected to staining of viral NSP4, as visualized by fluorescent microscopy.

### Results

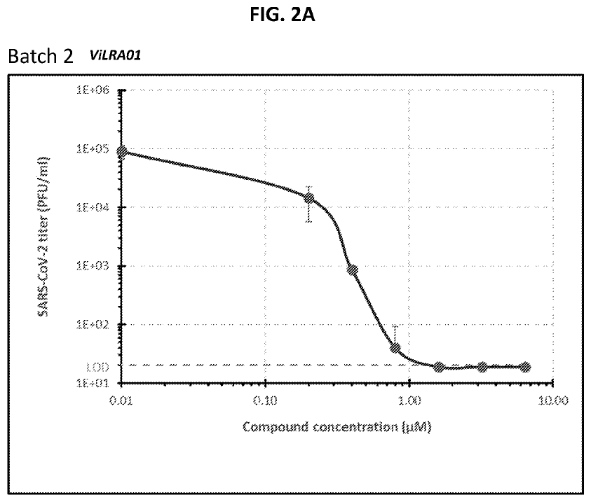

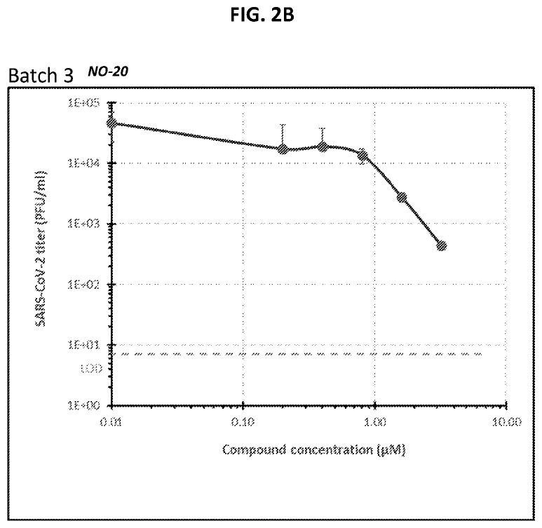

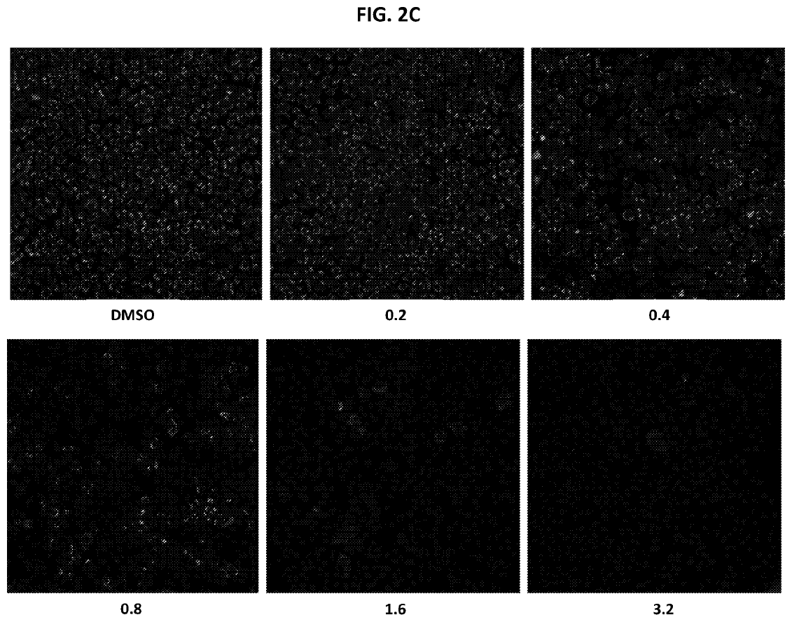

As shown in FIGS. 2A-2C, voclosporin inhibited SARS-CoV-2 viral load in a dose-dependent manner in Vero E6 cells and Calu cells. Batch 2 voclosporin inhibited SARS-CoV-2 viral load in E6 cells in a dose-dependent manner from 0.01 to 1.00 and as concentration increased above 1.00 viral titer was reduced to the limit of detection (FIG. 2A). Similarly, batch 3 voclosporin inhibited SARS-CoV-2 viral load in E6 and human Calu cells, respectively in a dose-dependent manner from 0.01 to 4.00 μM (FIG. 2B). As shown in FIG. 2C, voclosporin reduced the amount of SARS-CoV-2 infected cells in a dose-dependent manner, as reflected by the reduction in fluorescent staining of viral NSP4.

### Example 3

To evaluate the efficacy and safety profile of voclosporin treatment for preventing rejection in kidney transplant patients, de novo kidney transplant patients were administered with either voclosporin or tacrolimus, and assessed for rejection and other adverse outcomes.

### Methods

De novo kidney transplant recipients were enrolled into a 6 month, Phase 2b, multi-center, randomized, open-label study. The de novo kidney transplant recipients were administered twice daily (BID) with high-dose voclosporin (0.8 mg/kg), mid-dose voclosporin (0.6 mg/kg), low-dose voclosporin (0.4 mg/kg), or standard dose of tacrolimus (0.05 mg/kg). In addition, all subjects received induction immunosuppression with intravenous daclizumab or basiliximab (dosed per product labeling) and received concomitant treatment during the study with MMF and corticosteroids. The blood trough level of voclosporin or tacrolimus was measured over the course of 180 days. Adverse reactions were recorded and renal graft rejection was assessed based on Banff classification. At 3-month and 6-month time points, adverse reactions were analyzed. Graft survival and patient survival were also recorded at the 6-month time point.

### Results

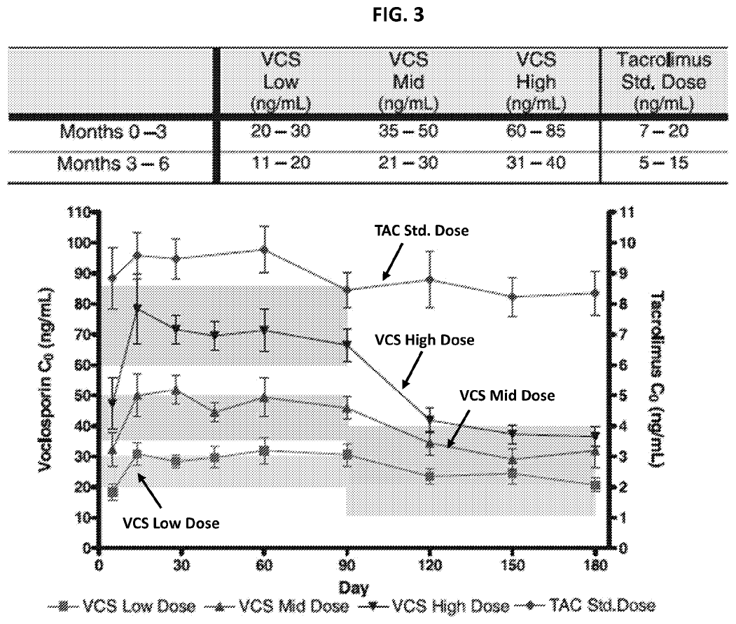

As shown in FIG. 3, blood trough levels (Co) for voclosporin for the low-dose, mid-dose and high-dose groups were 20-30 ng/mL, 35-50 ng/mL and 60-85 ng/mL respectively for months 0-3; and 11-20 ng/mL, 21-30 ng/mL and 31-40 ng/mL correspondingly for months 3-6. In comparison, the blood trough level for tacrolimus standard dose is 7-20 ng/mL for months 0-3; and 5-15 ng/mL for months 0-6.

The clinical impact of voclosporin administration was assessed by rates of graft rejection. As shown in Table 1, the mid-dose voclosporin group (0.6 mg/kg BID) displayed similar rejection rates as compared to the standard dose tacrolimus group, as shown by biopsy proven acute rejection (BPAR). The incidences of new onset of diabetes after transplant (NODAT) for low-dose, mid-dose and high-dose voclosporin groups were respectively 1.6%, 5.7% and 17.7%, compared to 16.4% for tacrolimus standard dose group. Nankivell eGFR (an indicator of renal function) for the low-dose, mid-dose and high-dose voclosporin groups were respectively: 71, 72 and 68 mL/min compared to 69 mL/min for tacrolimus standard dose group. This 6-month study showed that VCS is as efficacious as TAC in preventing acute rejection with similar renal function in the low and medium dose groups, and potentially associated with a reduced incidence of NODAT.

TABLE 1
Incidence of acute rejection at 6 month post-transplantation
under voclosporin or tacrolimus treatment

| | Voclosporin | Voclosporin | Voclosporin | Tacrolimus |
|---|---|---|---|---|
| | low dose | mid dose | high dose | standard dose |
| | (N = 84) | (N = 77) | (N = 87) | (N = 86) |
| Central | 9 (10.7%) | 7 (9.1%) | 2 (2.3%) | 5 (5.8%) |
| BPAR | | | | |
| NODAT | 1.6% | 5.7% | 17.7% | 16.4% |
| Graft | 100% | 100% | 98.9% | 97.7% |
| survival | | | | |
| Patient | 100% | 100% | 98.9% | 97.7% |
| survival | | | | |

### Example 4

Pharmacokinetic data was collected from a study on patients with active lupus nephritis. Trough concentrations for various doses are shown in Table 2. PK demonstrate the linearity of dose to trough concentration, such that the ongoing clinical trials do not require the use of therapeutic drug monitoring.

TABLE 2
Dose Trough Concentrations

| Dose | Trough concentration |
|---|---|
| 39.5 mg po BID | 32.7 ng/mL |
| 23.7 mg po BID | 21.1 ng/mL |
| 7.9 mg po BID | 7.3 ng/mL |

### Example 5

To evaluate the anti-viral effects of voclosporin in SARS-CoV-2 positive kidney transplant patients, kidney transplant recipients displaying mild to moderate SARS-CoV-2 symptoms are enrolled in a study to assess the effect combination therapy of prednisone and tacrolimus.

### Methods

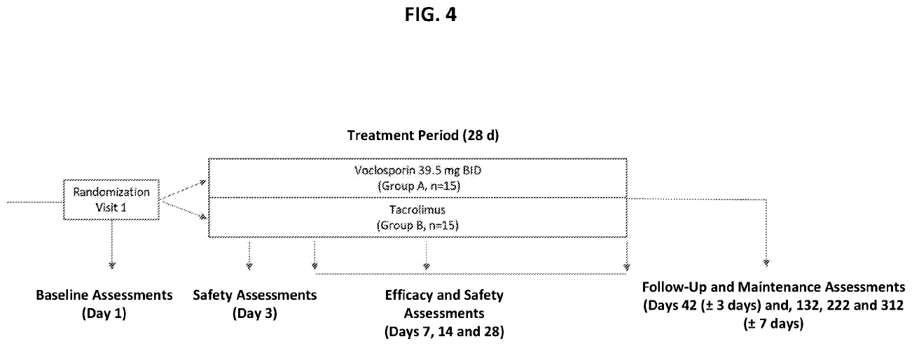

SARS-CoV-2 positive kidney transplant patients are enrolled in an open-label, single-center, exploratory study for voclosporin treatment. Prior to or at study entry, subjects have their standard immunosuppressive therapy reduced to dual therapy with prednisone and tacrolimus according to current local guidelines (LUMC Transplant Center treatment guidelines for COVID-positive transplant patients). Prior to Day 1, kidney transplant recipients with suspected COVID-19 infection have a SARS-CoV-2 diagnostic test and are informed about the study. Once COVID-19 infection has been confirmed, and upon consent, subjects are randomized into study groups and have Day 1 study procedures performed accordingly. Specifically, 15 out of 30 subjects remain on the therapy of prednisone and tacrolimus for the duration of the study, while the other 15 subjects switch from tacrolimus to voclosporin. Voclosporin is given as 6 capsules (of 7.9 mg each) BID for a treatment period of up to 1 year (FIG. 4). Safety drug monitoring take place during the study to ensure that voclosporin trough levels are maintained between 25-60 ng/mL and tacrolimus trough levels are maintained between 3-7 ng/ml. If trough levels are not within these levels, dose adjustments take place.

From Day 2-14, subjects undergo daily home-monitoring; and from Day 16-28 they undergo home-monitoring once every other day. Home-monitoring takes place via video consulting and includes self-measurements of temperature, blood pressure, pulse, weight, breathing frequency and oxygen saturation, as readouts of cytopathic effects (CPEs) by SARS-CoV-2. In addition, subjects are collecting first morning throat swabs for assessment of viral loads.

Subjects are also scheduled for 4 clinic visits, on Day 4 (Visit 2), Day 7 (Visit 3), Day 14 (Visit 4) and Day 28 (Visit 5/End of Study/Early Termination Visit) (FIG. 4). First morning throat swabs are also collected during visits. After Day 28 subjects continue in an extended safety follow-up with visits taking place at Day 42, 90, 180, 270 and 360 for maintenance of assessments. Study medication is dispensed to those subjects who choose to continue voclosporin after Visit 5 for up to 1 year.

### Results

Subjects placed on therapy of prednisone and tacrolimus and subjects placed on therapy of prednisone and voclosporin have their SARS-CoV-2 viral titers as well as cytopathic effects compared. Subjects in the voclosporin group are expected to have more efficacious reduction in viral load, and less severe CPEs compared to the tacrolimus group.

Subjects placed on therapy of prednisone and tacrolimus and subjects placed on therapy of prednisone and voclosporin are also compared for graft rejections, new onset of diabetes after transplant and other adverse effects. Subjects in the voclosporin group are expected to have comparable or lower incidences of graft rejections, diabetes and adverse effects when compared to the tacrolimus group.

### Example 6

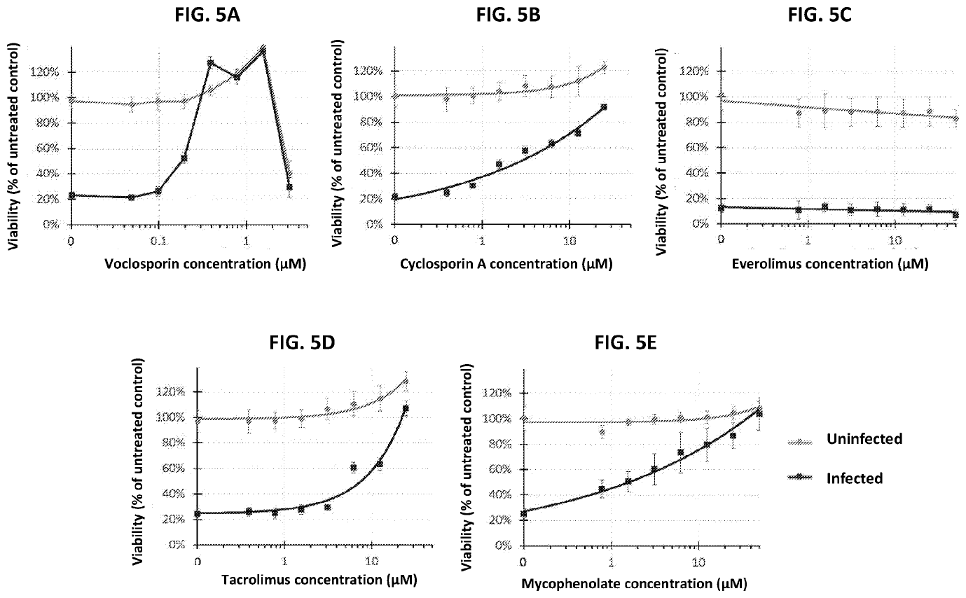

Voclosporin (Aurinia), cyclosporin (Novartis), tacrolimus (Astellas), mycophenolate (Roche) and everolimus (Novartis) stock solutions were prepared by dissolving the pharmaceutical formulation of these drugs in DMSO (therefore concentrations in FIGS. 5A-5E are estimated concentrations). Vero E6 cells (˜20,000 cells/well) in 96-well cell culture plates were infected with SARS-CoV-2 (multiplicity of infection 0.015), followed by incubation in 150 μl of medium with serial dilutions of the immunosuppressive agents. Virus-induced cell death was quantified three days post-infection by MTS assay and absorption was measured at 495 nm. Viability of noninfected cells was assessed in parallel to determine drug cytotoxicity. Two independent experiments (in quadruplicate) were performed for each drug. The 50% effective concentration (EC₅₀), defined as the concentration that inhibits virus-induced cell death by 50%, and the 50% cytotoxic concentration (CC₅₀), the concentration that reduces viability of uninfected cells to 50% of that of untreated control cells, were determined using non-linear regression with GraphPad Prism v8.0. As shown in FIGS. 5A-5E, only voclosporin (FIG. 5A), cyclosporin (FIG. 5B), tacrolimus (FIG. 5D) and mycophenolate (FIG. 5E) inhibited virus-induced cell death with EC₅₀ values of 0.27, 3.2, 12 and 3.1 μM, respectively.

Tacrolimus (FIG. 5D) and cyclosporin's (FIG. 5B) EC₅₀ concentrations are likely toxic at corresponding concentrations in vivo. However, as shown in FIG. 5A, voclosporin maintained cell viability and inhibited SARS-CoV-2 viral replication at approximately 40-fold and 10-fold lower concentrations than tacrolimus and cyclosporin respectively. Voclosporin's EC₅₀ is within the range of the Cₘₐₓ observed in transplant patients.

### Example 7: Inhibition of SARS-CoV-2 Replication in Calu-3 Cells by VCS, CsA and TAC

CPE reduction assays and virus yield reduction assays were performed to evaluate the effect of three calcineurin inhibitors cyclosporine A (CsA), tacrolimus (TAC), and voclosporin (VCS) against and other immunosuppressants commonly used in kidney transplant recipients (KTRs) on SARS-CoV-2 replication using cell-based assays.

### Methods

### Virus and Cell Lines

SARS-CoV-2/Leiden-0002 (GenBank MT510999) was isolated from a nasopharyngeal sample at Leiden University Medical Center (LUMC) in March 2020. Infections were performed with a virus stock that had been passaged twice in Vero E6 cells. Vero E6 cells and Calu-3 2B4 cells (Tseng et al., J Virol. August 2005; 79(15):9470-9), referred to as Calu-3 cells herein, were cultured as described previously (Salgado-Benvindo et al., Antimicrob Agents Chemother. Jul. 22 2020; 64(8) doi:10.1128/AAC.00900-20). Infections were performed in Eagle's minimal essential medium (EMEM; Lonza) with 25 mM HEPES (Lonza), 2% FCS, 2 mM L-glutamine, and antibiotics (EMEM-2% FCS). All experiments with infectious SARS-CoV-2 were performed in a biosafety level 3 facility at the LUMC.

### Immunosuppressive Compounds

Voclosporin (VCS; Lupkynis™), cyclosporine A (CsA; Neoral®, Novartis), tacrolimus (TAC; Prograf®, Astellas), mycophenolate mofetil (MMF; CellCept®, Roche) or everolimus (EVL; Certican®, Novartis) stock solutions were prepared by dissolving the pharmaceutical formulation of these drugs in dimethyl sulfoxide (DMSO). Placebo capsules and pure VCS powder, Tacrolimus (PHR1809), cyclosporin A (30024) and mycophenolic acid (M5255) were obtained. Remdesivir (RDV; HY-104077) was used as a control in all experiments. All compounds were dissolved in DMSO and single use aliquots were stored at −20° C.

### Measurement of Cyclosporin a, Tacrolimus and Voclosporin Concentrations by Validated LC-MS/MS

Quantification of CsA and TAC was performed by LC-MS/MS as previously described (Zwart et al., Br J Clin Pharmacol. December 2018; 84(12):2889-2902) by diluting samples in methanol and subsequently in blank whole blood. Before analysis, samples were diluted in methanol and subsequently whole blood to fall within the calibration line of 0-15-600 m/L of VCS. Human whole blood was added to 10- or 20-μl samples to a final volume of 200 μl and 200 μl of 0.1 M zinc-sulphate and 500 μl of internal standard solution (32 μg/L of VCS D4 in acetonitrile) were added. Samples were then vortexed at 2000 rpm for 5 min and centrifuged at 13000 rpm for 5 min and 20 μl was injected into the LC-MS/MS system. The method was validated according to the EMA bioanalytical method validation guideline (EMEA/CHMP/EWP/192217/2009—Guideline on bioanalytical method validation (2011)).

Cytopathic effect (CPE) reduction assay

CPE reductions assays in Vero E6 cells were performed, generally as described above, except that pre-incubation of cells with the tested compounds lasted 30 minutes. Plates were incubated for three days at 37° C. and cell viability was determined using a colorimetric assay, measuring the absorption at 495 nm. The EC₅₀ and CC₅₀ of each compound was determined and analysis of the resultant data using non-linear regression. For each compound, at least two independent experiments (each in quadruplicate) were completed.

### Virus Yield Reduction Assay

Calu-3 cells were seeded in 96-well plates (3×10⁴ cells per well) in 100 μl of culture medium. The next day, cells were pre-incubated for 60 min with 2-fold serial dilutions of CsA, TAC or VCS, starting at 25 μM concentration and RDV starting at 10 μM. Subsequently, cells were infected with SARS-CoV-2 (MOI of 1, based on titer determined on Vero E6 cells) in 50 μl of medium with compound. After a 1 h incubation at 37° C., cells were washed three times with PBS and 100 μl of medium with compound was added. The medium was harvested from the wells at 24-hours post-infection (h p.i.). Analysis of viral progeny released from the infected Calu-3 cells was performed by plaque assay on Vero E6 cells. VCS concentrations were measured by validated LC-MS/MS after adding 9 volumes of methanol to the harvested medium. A cytotoxicity assay with mock infected cells, treated in the same way, was performed in parallel, as described for the CPE reduction assay.

### Virus Yield Reduction in Glass Bottles

Borosilicate glass reagent bottles (50-ml) were treated with glacial acetic acid to remove possible detergent residues, followed by washing twice with absolute ethanol. The bottles were dried and UV-sterilized prior to use. Three times concentrated compound solutions were prepared in EMEM-2% FCS using sterile glass culture tubes, a glass 50-μl syringe and glass Pasteur pipettes. One ml of each compound dilution was transferred to three different reagent bottles (triplicates). Confluent monolayers of Calu-3 cells grown in culture flasks were infected with SARS-CoV-2/Leiden-002 at an MOI of 1. After incubation for 1 h at 37° C., cells were washed three times with warm PBS, trypsinized and resuspended in EMEM-2% FCS. Two ml of this cell suspension (˜10⁶ cells) was added to each reagent bottle that contained 1 ml of a 3× concentrated compound solution in medium. After incubation for 24 h at 37° C., the medium was collected and the infectious virus titer was determined by plaque assay on Vero E6 cells.

### Determination of Compound Cytotoxicity in Glass Culture Tubes

Calu-3 cells were trypsinized and 1.5×10⁵ cells in 1 ml of EMEM-2% FCS were divided over glass culture tubes. Two-fold dilutions of VCS, TAC and CsA starting at 150 μM concentration (3× final concentration) were prepared in EMEM-2% FCS medium using glass labware, and 0.5 ml was added to corresponding tubes with cells (three tubes per concentration). After a 24 h incubation, cell viability was determined as described above.

### Results

To evaluate the effect of VCS, CsA and TAC on SARS-CoV-2 replication, viral load reduction assays were performed using human lung epithelial cells (Calu-3), shown to be permissive to SARS-CoV-2. As VCS is highly lipophilic and could bind to plastic, which could reduce the bioavailability in assays using plastic labware, the effect of VCS was compared in standard cell-based assays using plastic labware and custom assays using glass tubes, containers and pipettes. RDV was included as a positive control for inhibition of SARS-CoV-2 replication.

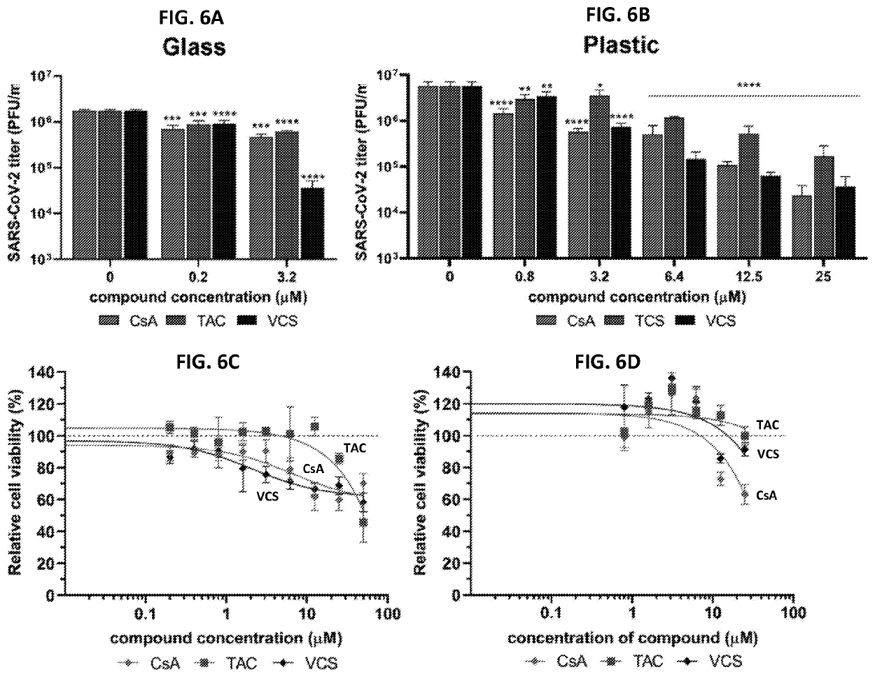

FIGS. 6A-6D show the impact of cyclosporine A, tacrolimus, and voclosporin on the production of infectious SARS-CoV-2 progeny after infection (FIG. 6A and FIG. 6B) and the viability Calu-3 cells after mock infection (FIG. 6C and FIG. 6D). FIG. 6A and FIG. 6C show data derived from experiments performed using glass labware, while FIG. 6B and FIG. 6D show results from use of plastic labware. Calu-3 cells in glass remained viable and supported SARS-CoV-2 replication, as titers of 1.7×10⁶ PFU/ml were measured in the medium at 24 h p.i. (FIG. 6A). Treatment of infected cells with 10 μM of remdesivir inhibited viral replication resulting in infectious progeny titers just above the limit of detection of the plaque assay (data not shown). Treatment of cells with 3.2 μM voclosporin caused a more than 1.5 log reduction in SARS-CoV-2 infectious progeny titers, while an ˜0.5 log reduction was observed when the same concentration of cyclosporin A or tacrolimus was used (FIG. 6A). However, treatment with 3.2 μM voclosporin or cyclosporin A also caused cytotoxic effects, as cell viability dropped to ˜75% (FIG. 6C). Taken together, these results demonstrate potent antiviral activity of voclosporin, which may be in part mediated by cytotoxicity.

The observed results were consistent with a previous report that CsA inhibited SARS-CoV-2 replication in HuH7.5 and Calu-3 cells, but not in Vero cells (Dittmar et al., bioRxiv. 2020:2020.06.19.161042). However, in contrast to the observations that TAC inhibits SARS-CoV-2 replication in Vero E6 cells with an EC₅₀ of ˜15 μM, the previous report found no activity for TAC in any of these cell lines, which may be contributed to the use of different Vero cell subclones.

In experiments using plastic materials, a dose-dependent reduction in infectious progeny titers was observed when cells were treated with VCS, leading to a more than 1 log reduction at 6.4 μM (FIG. 6B). CsA treatment led to a similar reduction at 25 μM, but at 6.4 μM inhibited less than VCS. However, at concentrations of 12.5 μM or above CsA displayed significant cytotoxicity while VCS did not (FIG. 6D). TAC did not display much cytotoxicity, but a concentration of 25 μM was required to reduce the infectious virus progeny titer by more than 1 log. VCS had a stronger effect in experiments performed with glass instead of plastic labware, likely due to loss of the compound binding to plastic. The concentration of free VCS was measured after incubation with various solutions in glass containers, either with or without cells. No significant loss of compound from solution was observed after a 24 h incubation at 37° C. in glass without cells (Table 3). When VCS solutions with concentrations from 0.2 to 3.2 μM were incubated in glass bottles with Calu-3 cells, a ˜75% reduction of the VCS concentration was observed, indicating that the compound was bound or taken up by cells. The VCS concentration in the medium of infected cells after 24 h treatment with 25 μM VCS in experiments performed with standard plastic labware was also measured, and the detected concentration of voclosporin was as low as 0.68 μM.

TABLE 3
VCS concentration in samples from experiments
using only glass labware, measured by LC-MS/MS

| | Concentration of VCS in supplied solution | | | | | | | | | | | |
|---|---|---|---|---|---|---|---|---|---|---|---|---|
| | 3.2 μM | | 3.2 μM | | 1.6 μM | | 0.8 μM | | 0.4 μM | | 0.2 μM | |
| | Without cells | | With cells | | | | | | | | | |
| Incubation | Conc | % | Conc | % | Conc | % | Conc | % | Conc | % | Conc | % |
| time | (μM) | rem | (μM) | rem | (μM) | rem | (μM) | rem | (μM) | rem | (μM) | rem |
| 0 h | 2.91 | | 2.91 | | 1.77 | | 0.99 | | 0.45 | | 0.33 | |
| 24 h | 2.79 | 96 | 0.82 | 28 | 0.35 | 20 | 0.15 | 15 | 0.10 | 22 | <0.07* | ND |

*Below detection limit of LC/MS-MS: The percentages indicate the ratio of the measured (true) concentration at 24 h and the concentration of the prepared solution administrated to the cells (at 0 h incubation time); % rem: % remaining

Even considering a 75% reduction due to cellular binding or uptake, these results indicate that 90% of VCS was lost due to plastic binding. The similar reduction in virus titers by 3.2 and 25 μM of VCS in glass and plastic, respectively, corroborated that when using plastic, the bioavailable amount of VCS is likely ˜10% of what was added initially. VCS binding to plastic can cause a >80% loss of the compound from solution. Consequently, the use of stock solutions prepared from pure VCS powder using plastic labware can be an underestimation of the compound's efficacy in antiviral assays. As VCS is a highly lipophilic compound, and interactions between plastic surfaces and hydrophobic drugs can have a negative effect, the antiviral effect of VCS is likely greater than observed in assays using plastic. The results indicate that loss of compound due to plastic binding and interference of excipients in pharmaceutical formulations complicated the determination of EC₅₀ values in antiviral assays that included use of plastic.

Using glass labware, the results showed demonstrated that VCS reduced the production of SARS-CoV-2 infectious progeny in a dose-dependent manner in infected Calu-3 cells, and more effectively than CsA and TAC. The results show that cyclophilin-dependent CNIs inhibit SARS-CoV-2 replication in cell culture more potently than other classes of immunosuppressants, such as EVL and MPA. VCS inhibited SARS-CoV-2 replication at 8-fold lower concentrations than TAC. TAC concentrations that are required to inhibit SARS-CoV-2 replication likely are intolerable or toxic concentrations in humans (EC₅₀ of 0.2 μM equals 160 ng/ml for TAC), without taking into account that the free fraction in traffic is around one tenth of the total concentration. For CsA and VCS, 0.2 μM corresponds to a concentration of 241 and 243 ng/ml respectively. Notably, VCS can distribute into organs such as the lungs in higher concentrations than in blood, and higher concentrations are found in red blood cells. Consequently, higher concentrations in specific organs or cells can inhibit the virus. Accordingly, the results support the utility of VCS as a CNI for therapy that also can inhibit SARS-CoV-2 replication at concentrations that are safe in humans. As VCS is thought to have comparable efficacy to TAC for prevention of rejection in KTRs, VCS would be useful in treatment for COVID-19 patients. The results described herein demonstrate a benefit of cyclophilin-dependent CNIs, in particular VCS, among immunosuppressants commonly used in transplant medicine, for subjects in need of immunosuppression, for example, KTRs, that are at risk of a SARS-CoV-2 infection.

### Example 8: Inhibition of SARS-CoV-2 Replication by Pharmaceutical Formulations of Immunosuppressive Agents

Experiments to evaluate cell viability in response to SARS-CoV-2 infection were performed with pharmaceutical formulations of drugs commonly used to treat kidney transplant recipients. In view of the findings from Example 7, it was suspected that issues related to solubility or plastic binding may be avoided with use of pharmaceutical formulations containing, for example, solvents and excipients.

### Methods

Pharmaceutical formulations (including excipients, co-solvents and other components) of VCS, CsA, TAC, EVL, and MMF were evaluated in a CPE reduction assay using Vero E6 cells, as described above. Remdesivir (RDV; HY-104077) was used as a control in all experiments. Cells were kept in medium containing drug for a period of 3 days following pre-incubation and infection with SARS-CoV-2. As above, assays with mock-infected cells were performed in parallel to assess cytotoxicity of the tested formulations. For VCS, achievement of the concentration when dissolving stock solutions at 6.4 μM was confirmed by LC-MS/MS (data not shown).

### Results

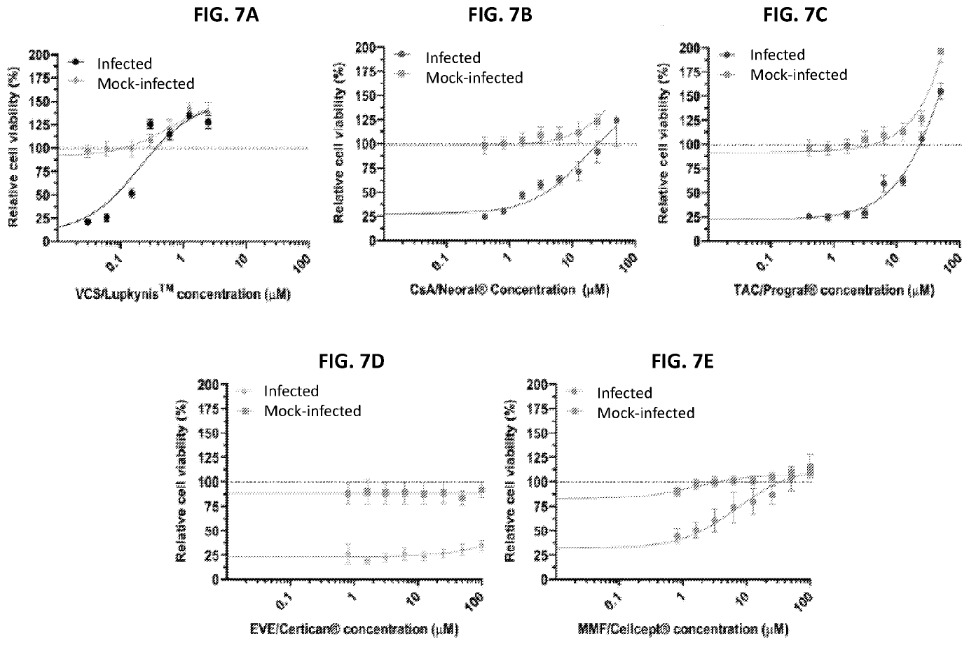

FIGS. 7A-7E show the effects of the various pharmaceutical formulations on cell viability in infected and mock-infected cells. The CNIs VCS, CsA, and TAC inhibited virus-induced cell death with EC₅₀ values in the sub to low micromolar range (FIGS. 7A-7C). EVL (FIG. 7D) did not show an inhibitory effect at the tested concentrations. The prodrug MMF (FIG. 7E) was included in the comparison, but was not expected to inhibit virus replication, as it is likely not metabolized into its active form mycophenolate (also known as mycophenolic acid; MPA) (Ransom, Ther Drug Monit. 1995; 17(6):681-4) during the assay (Ritter and Pirofski, Transp Infect Dis. 2009; 11(4):290-7 and Neyts et al., Antimicrob Agents Chemother. 1998; 42(2):216-22). Thus, apparent antiviral effect of MMF may be attributed to excipients present in the drug formulation.

The EC₅₀ values of VCS, CsA and TAC were 0.22±0.01 μM, 4.3±0.6 μM and 10±1 μM, respectively. Apart from VCS, none of the compounds showed cytotoxicity, and therefore their CC₅₀ values were higher than 100 μM. Although VCS displayed higher cytotoxicity, with a CC₅₀ of ˜4 μM, its EC₅₀ was also 18-45 times lower compared to the other compounds tested. These results demonstrate the superior effects of VCS on inhibiting viral-mediated cell death, as compared to the other tested compounds.

### Example 9: The Effect of a Pharmaceutical Formulation of VCS on SARS-CoV-2 Replication

Assays were performed to assess the antiviral effect of the content of VCS capsules and placebo capsules, for example, to determine whether one or more excipients in Lupkyna™ (VCS pharmaceutical formulation) contributed to the effect of VCS, for example, the low EC₅₀ of ˜0.22 μM as shown in Example 8 (FIG. 7A).

### Methods

CPE reduction assays were performed as described above. Vero E6 cells infected with SARS-CoV-2 were exposed to VCS or placebo to compare the antiviral (infected cells) and cytotoxic (mock-infected cells) effects of each. The absence of VCS in placebo capsules was confirmed by LC-MS/MS analysis (data not shown).

### Results

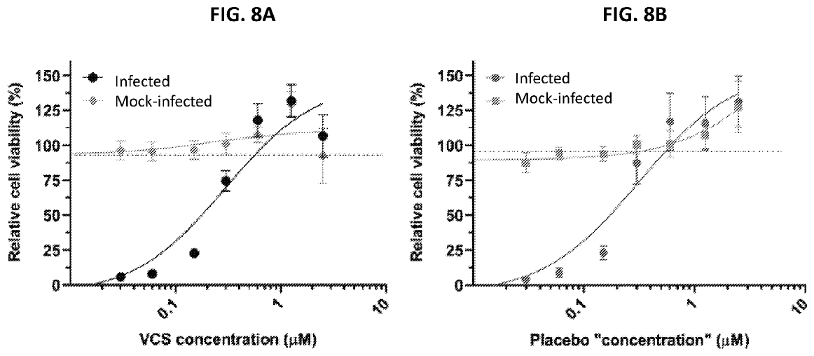

FIGS. 8A-8B compare the effects of a VCS pharmaceutical formulation and a placebo on cell viability. Surprisingly, both the VCS formulation (FIG. 8A) and the placebo (FIG. 8B) inhibited SARS-CoV-2 replication in a similar dose-dependent manner. These results indicated that one or more excipients in the VCS drug formulation could mediate antiviral activity under the described experimental conditions.

### Example 10: Assessment of Virucidal Effects of Formulation of VCS

Potential virucidal activity of the placebo capsule was further assessed.

### Methods

To determine the virucidal potential of compounds or formulations, SARS-CoV-2 virions (5×10⁴PFU) were incubated for 2 h at 37° C. with one of the following solutions: medium, a VCS solution prepared from pure powder (3.2 μM), the dissolved content of VCS capsules (3.2 μM), placebo capsules or Tween solutions (present in the capsules, corresponding to 3.2 μM VCS). PBS was used as a negative control and 50% ethanol as a positive control for virucidal activity. Tested compounds were incubated with a SARS-CoV-2 virus stock for two hours. The remaining infectious virus titer was determined by plaque assay on Vero E6 cells as described in, for example, Salgado-Benvindo et al., Antimicrob Agents Chemother. Jul. 22 2020; 64(8) doi:10.1128/AAC.00900-20.

### Results

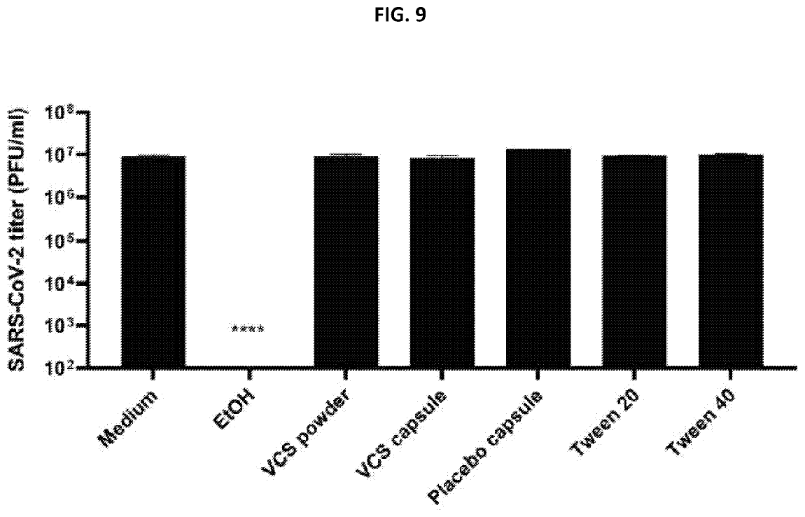

As shown in FIG. 9, only the control treatment (50% ethanol) reduced the amount of infectious SARS-CoV-2 to below the limit of detection (<100 PFU/ml). None of the other treatments significantly affected the remaining infectivity of the virus.

The results showed that the drug product excipients had no virucidal effect under the described experimental conditions. The results indicated a possibility that the presence of an excipient, through an uncharacterized mechanism, interfered with the readout of the CPE reduction assays.

### Example 11: Preparation of Immunosuppressive Compounds from High Purity Powders and their Activity in CPE Reduction Assays

The effect of excipients in pharmaceutical compositions on the effect of VCS and other immunosuppressive compounds were assessed.

### Methods

CPE reduction assays were performed as described above, using high purity powders of immunosuppressive drugs solubilized in DMSO. Test stock solutions were prepared from pure powders of each compound. In the case of Neoral (CsA microemulsion), CsA powder, the most commonly used CsA derivative in KTR treatment, was evaluated.

### Results

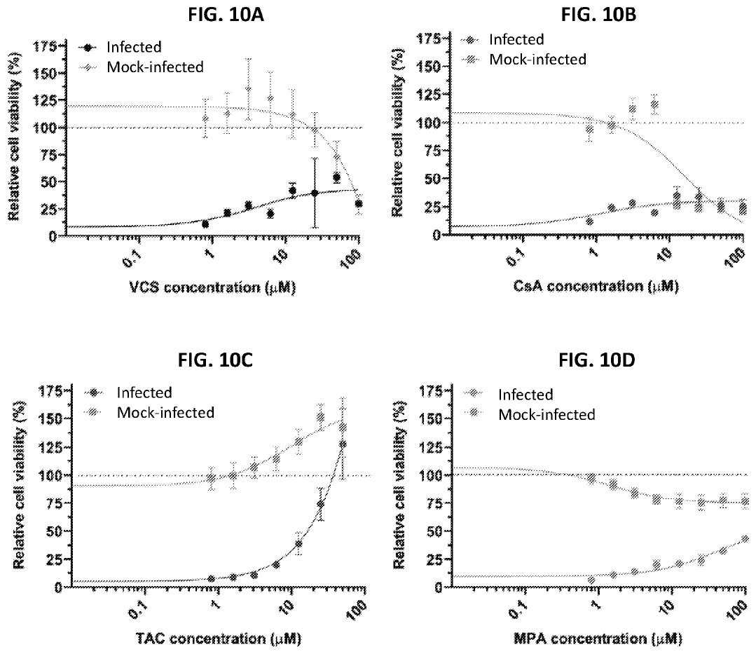

FIGS. 10A-10D show the effects of solubilized high purity immunosuppressive compounds on cell viability in a CPE reduction assay. As shown in FIG. 10A, VCS solutions prepared from pure powder did not confer the same level of protection to SARS-CoV-2 infected-cells as solutions made from the pharmaceutical formulation (compare FIG. 8A). However, the VCS solution from pure powder also caused less cytotoxicity, as observed from mock-infected cells (compare FIG. 6C). FIGS. 10B and 10D show similar results for CsA and MPA treated cells, respectively. When comparing FIG. 7C to FIG. 10C, TAC solutions prepared from pure powder inhibited SARS-CoV-2 with similar efficacy as the drug formulations, i.e., with an EC₅₀ of ˜15 μM.

Together, these results indicate that the excipients of certain pharmaceutical formulations contribute to their observed efficacy. Testing of highly pure powders of the various immunosuppressive compounds to circumvent the interference caused by excipients in the antiviral assays, resulted in substantially higher EC₅₀ values for VCS, CsA and TAC, demonstrating that excipients that improve solubility and bioavailability of the active compound in pharmaceutical formulations could also affect results in cell-based assays. However, the pharmaceutical formulation of TAC did not appear to contain excipients with antiviral effects.

The results show that immunosuppressive compounds may need excipients to ensure solubility and/or bioavailability for optimal activity. The preparation of the compound, for example, pharmaceutical formulation or solubilized high purity powder, can impact results in assays and the solubility and bioavailability of the compounds when administered.

### Example 12: Assessment of the Effect of Plastic Materials in the Formulation of Voclosporin

In view of the potential interaction between VCS and plastic labware, experiments were performed to determine whether coating plastic would prevent VCS binding. As described above, the excipients in the pharmaceutical formulation of VCS could affect the bioavailability, for example by preventing binding of VCS to plastic, but their non-specific antiviral effects may also affect the determination of the true EC₅₀ of VCS. Plastic material coated with different agents were tested to assess whether the coating would prevent VCS from binding to plastic, and potentially allow the use of VCS solutions prepared from pure powder in antiviral assays.

### Methods

### Coating of Plastic Materials

Plastic labware was coated with three different coating agents: 100 mg/ml bovine serum albumin in PBS (BSA; Sigma), 1% polyethylene glycol 3350 in MilliQ water (PEG-3350; Sigma) and 0.2% polysorbate 40 in MilliQ water (Tween40; Fluka). In addition, the plastic materials with VCS by treating them with a 500 mM VCS solution in DMSO (Sigma). Labware, including all tubes, tips and culture plastics, was filled with blocking solution and incubated for 2 h at room temperature with rocking to homogenously coat the surfaces. After rinsing twice with MilliQ water, the items were left to dry at room temperature until further use in experiments. Solutions of 0.2 and 2 μM of VCS were prepared in EMEM-2% FCS and 100 μl of each VCS solution was incubated in coated 96-well plates. After a 2 h incubation at 37° C. the remaining VCS concentration was measured by validated LC-MS/MS. Using similar methods, the binding of TAC or CsA was also assessed.

### Results

None of the coating treatments were able to reduce the nonspecific binding to plastic and loss of VCS (see Table 4), as only 5 to 7% of the original concentration was recovered after a 2 h incubation. Even at t=0 only ˜27% of the original stock concentration could be recovered due to VCS loss in pipette tips and tubes during the preparation of dilutions. Saturation of binding sites on plastic by treatment with 500 mM of VCS prevented loss of VCS from solution, but led to non-controlled VCS leaching from the plastic. This resulted in unpredictable concentrations that were higher than those in the input solution, e.g., a VCS concentration of >15 μM was measured when a 2 μM solution was incubated in a VCS saturated plastic plate.

TABLE 4
VCS concentration in samples incubated in plastic labware with different coatings, measured by LC-MS/MS

| | Type of coating applied | | | | | | | | | |
|---|---|---|---|---|---|---|---|---|---|---|
| | | | | | 100 mg/ml BSA | | 1% PEG-3350 | | 0.2% Tween-40 | |
| | Uncoated | | 500 mM VCS | | solution | | solution | | solution | |
| Incubation | Conc | % | Conc | % | Conc | % | Conc | % | Conc | % |
| time | (μM) | rem | (μM) | rem | (μM) | rem | (μM) | rem | (μM) | rem |
| 0 h | 0.56 ± 0.25 | 28 | 17.21 ± 2.36 | 861% | 0.55 ± 0.21 | 27% | 0.51 ± 0.16 | 26% | 0.56 ± 0.35 | 28% |
| 2 h | 0.13 ± 0.07 | 7 | 2.73 ± 1.00 | 137% | 0.10 ± 0.04 | 5% | 0.09 ± 0.02 | 4% | 0.09 ± 0.04 | 4% |

Conc: concentration; % rem: % remaining. The percentages indicate the remaining concentration relative to the concentration of the original 2 μM of VCS stock solution.

Binding to plastic was minimal for TAC (24% loss). For CsA, after a 2 h incubation, the remaining concentration was 62% of the initial concentration (see Table 5).

TABLE 5
Concentration of TAC and CsA in samples incubated
in plastic labware, measured by LC-MS/MS

| | |
|---|---|
| TAC | CsA |

| Incubation | Conc | | Conc | |
|---|---|---|---|---|
| time | (μM) | % rem | (μM) | % rem |
| 0 h | 0.85 | | 0.76 | |
| 2 h | 0.65 | 76 | 0.47 | 62 |

The percentages indicate the remaining concentration relative to the concentration of the original compound stock solution (0.8 μM).

The results showed that coating agents did not prevent nonspecific binding of VCS to plastic. In some aspects, loss of compound due to plastic binding and interference of excipients in pharmaceutical formulations complicated the determination of EC₅₀ values in some of the assays. As none of the coating treatments prevented nonspecific binding to plastic, glassware was used instead of plastic in other experiments (e.g., see Table 3) to circumvent potential issues.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention. Various modifications to the compositions and methods described will become apparent from the description and teachings herein. Such variations may be practiced without departing from the true scope and spirit of the disclosure and are intended to fall within the scope of the present disclosure.

1. A method of treating or preventing a virus infection in a subject, comprising administering to the subject a therapeutically effective amount of voclosporin.

2. The method of claim 1, wherein the subject is in need of immunosuppression.

3. The method of claim 1 or 2, wherein the virus infection is ameliorated by the inhibition of cyclophilin A (CypA) or a CypA associated pathway.

4. The method of any one of claims 1-3, wherein the virus infection is caused by a virus that is a member of Coronaviridae.

5. The method of claim 4, wherein the virus is an alphacoronavirus, a betacoronavirus, a deltacoronavirus, or a gammacoronavirus.

6. The method of claim 3 or 4 wherein the virus is Human coronavirus OC43 (HCoV-OC43), Human coronavirus HKU1 (HCoV-HKU1), Human coronavirus 229E (HCoV-229E), Human coronavirus NL63 (HCoV-NL63), Middle East respiratory syndrome-related coronavirus (MERS-CoV), Severe acute respiratory syndrome coronavirus (SARS-CoV), or Severe acute respiratory syndrome coronavirus 2 (SARS-CoV-2).

7. The method of any one of claims 3-6, wherein the virus is MERS-CoV, SARS-CoV, or SARS-CoV-2.

8. The method of any one of claims 3-7, wherein the virus is SARS-CoV-2.

9. The method of any one of claims 1-8, wherein the therapeutically effective amount is about 0.1 mg/kg/day to about 2 mg/kg/day.

10. The method of any one of claims 1-8, wherein the therapeutically effective amount is about 7.9 mg BID, about 15.8 mg BID, about 23.7 mg BID, about 31.6 mg BID, about 39.5 mg BID, about 47.4 mg BID, or about 55.3 BID.

11. The method of any one of claim 1-8, wherein the therapeutically effective amount is about 7.9 mg QD, about 15.8 mg QD, about 23.7 mg QD, about 31.6 mg QD, about 39.5 mg QD, about 47.4 mg QD, about 55.3 mg QD, about 63.2 mg QD, about 71.1 mg QD, about 79.0 mg QD, about 86.9 mg QD, about 94.8 mg QD, about 102.7 mg QD, or about 110.6 mg QD.

12. The method of any one of claim 1-8, wherein the therapeutically effective amount is equivalent to or can achieve a concentration of between about 0.05 μM and about 10 μM, about 0.1 μM and about 5 μM, about 0.2 μM and about 2.5 μM, about 0.3 μM and about 1.0 μM, about 0.4 μM and about 0.9 μM, about 0.5 μM and about 0.8 μM, about 0.1 μM and about 0.5 μM, or about 0.2 μM and about 0.4 μM, or about 0.05, about 0.1, about 0.15, about 0.2, about 0.25, about 0.3, about 0.35, about 0.4, about 0.45, about 0.5, about 0.55, about 0.6, about 0.7, about 0.8, about 0.9, about 1.0, about 1.5, about 2.0, about 2.5, about 3.0, about 3.5, about 4.0, about 4.5, about 5.0, about 6.0, about 7.0, about 8.0, about 9.0, or about 10.0 μM or less.

13. The method of any one of claims 1-12, wherein the renal function of the subject is monitored.

14. The method of claim 13, wherein monitoring the renal function of the subject comprises:
(a) assessing estimated Glomerular Filtration Rate (eGFR) of the subject at at least a first time point and a second time point on different days; and
(b) (i) if the eGFR of the subject decreases by more than a target % to below a predetermined value between the first and second time points, then reducing the daily dosage or stopping the administering of voclosporin to the subject;
(ii) if the eGFR of the subject decreases by less than the target % between the first and second time points, continuing administering the same daily dosage of voclosporin to the subject.

15. The method of claim 14, wherein the predetermined value is about 50 to about 90 ml/min/1.73 m².

16. The method of claim 14 or 15, wherein the predetermined value is about 60 ml/min/1.73 m².

17. The method of any one of claims 14-16, wherein the target % is about 20% to about 45%.

18. The method of any one of claims 14-17, wherein the target % is about 20%.

19. The method of any one of claims 1-18, wherein the subject has an autoimmune disease or a condition associated with transplant rejection.

20. The method of any one of claims 1-19, wherein the subject has a condition associated with transplant rejection.

21. The method of claim 19 or 20, wherein the condition is associated with heart, lung, liver, kidney, pancreas, skin, bowel, or cornea transplant rejection.

22. The method of any one of claims 19-21, wherein the condition is associated with kidney transplant rejection.

23. The method of claim 19, wherein the subject has an autoimmune disease.

24. The method of any one of claims 1-23, wherein the therapeutically effective amount of voclosporin is administered without administering a therapeutically effective amount of mycophenolate mofetil (MMF) and/or a therapeutically effective amount of a corticosteroid.

25. The method of any one of claims 1-23, further comprising administering a therapeutically effective amount of mycophenolate mofetil (MMF) and/or a therapeutically effective amount of a corticosteroid.

26. The method of any one of claims 1-25, wherein voclosporin is administered by enteral administration, oral administration, sublingual administration, or rectal administration, parenteral administration, intravenous injection, intramuscular injection, subcutaneous injection, intravenous infusion, or inhalation/insufflation.

27. The method of claim 26, wherein voclosporin is administered by enteral administration, oral administration, sublingual administration, or rectal administration.

28. The method of claim 25 or 27, wherein voclosporin is administered by oral administration.

29. The method of claim 26, wherein voclosporin is administered by parenteral administration, intravenous injection, intramuscular injection, subcutaneous injection, intravenous infusion, or inhalation/insufflation.

30. The method of claim 26 or 29, wherein voclosporin is administered by inhalation or insufflation.

31. The method of claim 30, wherein voclosporin is administered in the form of an aerosol.

32. The method of any one of claims 1-31, wherein voclosporin is administered in a pharmaceutical composition.

33. The method of claim 32, wherein the pharmaceutical composition comprises one or more pharmaceutically acceptable excipients.

34. The method of claim 33, wherein the pharmaceutically acceptable excipients are independently selected from one or more of comprising alcohol, D-α-tocopherol (vitamin E) polyethylene glycol succinate (TPGS), polysorbate 20 (Tween 20), polysorbate 40 (Tween 40), medium-chain triglycerides, gelatin, sorbitol, glycerin, iron oxide yellow, iron oxide red, titanium dioxide, and water.

35. The method of any one of claims 1-34, wherein the viral load is reduced in the subject following administration of voclosporin.

36. The method of any one of claims 1-35, wherein the survival of the subject is extended following administration of voclosporin.
